(19) United States
(12) Patent Application Publication
(10) Pub. No.: US 2012/0283295 A1
(43) Pub. Date: Nov. 8, 2012

(54) INDAZOLE DERIVATIVES AND THEIR USE FOR BLOCKADING VOLTAGE DEPENDENT SODIUM CHANNELS

(75) Inventors: Cristina Garcia Posada, London (GB); David Selwood, London (GB); John Garthwaite, London (GB); David Baker, London (GB); Lisa Clutterbuck, London (GB)

(73) Assignee: University College London, London (GB)

(21) Appl. No.: 13/499,517

(22) PCT Filed: Sep. 30, 2010
(86) PCT No.: PCT/GB10/01842

§ 371 Date: Jul. 13, 2012

(30) Foreign Application Priority Data
Oct. 1, 2009 (GB) 0917238.8

(51) Int. Cl. A61K 31/4245; C07D 417/14; A61K 31/427; A61P 29/00; A61P 27/06; A61P 25/00; A61P 25/28; A61P 25/16; C07D 413/14; A61K 31/4439

(52) U.S. Cl. 514/338; 548/131; 514/364; 546/269.1

(57) ABSTRACT

The invention provides an indazole derivative of formula (1), or a pharmaceutically acceptable salt or N-oxide thereof, wherein R¹, R², R³ and R⁴ are as defined herein. The indazole derivatives are capable of blockading voltage dependent sodium channels and they are useful in the treatment or prevention of normal tension glaucoma, multiple sclerosis, a motorneurone disease, stroke, spinal cord injury, Alzheimer's disease, Parkinson's disease or pain.

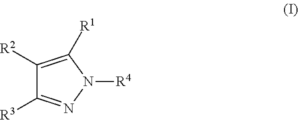

The present invention relates to a series of indazole derivatives capable of blockading voltage dependent sodium channels.

Voltage-dependent sodium channels are found in the cell membranes of neurones (including their axons) where they are fundamental to the generation and propagation of electrical impulses. Under pathological conditions (such as ischaemia), however, sodium channels become abnormally activated resulting in an excessive flow of sodium ions into the cytoplasm. The rise in cellular sodium ions then causes a large inflow of calcium ions leading to the activation of several mechanisms that lead to irreversible loss of function and subsequent degeneration.

It has already proved possible to produce pharmacological agents capable of stopping excessive activity of sodium channels without adversely affecting their normal function. Indeed, this is the principal mode of action of several widely-used and well-tolerated antiepileptic drugs (e.g. phenytoin, carbamazapine and lamotrigine). Sodium channel inhibitors have been shown to be protective towards neurones in the grey matter in several models of cerebral ischaemia. More recently, it has become evident that certain sodium channel inhibitors are highly effective in protecting axons in the in vitro optic nerve from irreversible damage imposed by severe deprivation of oxygen and glucose.

Significant evidence suggests that sodium channels are also involved in the activation of pain pathways (Wood, J. N. et al, (2005) Curr. Top. Med. Chem., 5, 529-537). As a result, many established sodium channel modulators, including anticonvulsants lamotrigine, carbamazepine, and phenytoin have been found to be effective for the treatment of chronic neuropathic pain. Further, it is also well-known that sodium channel modulators are capable of neuroprotection (Waxman, S. G. (2008) Nat Clin. Pract. Neurol., 4, 159-169). That will be useful for reducing brain damage following a stroke, where the brain is deprived of oxygen and glucose (Tymianski, M. et al (1996) Neurosurgery, 38, 1176-1195). Various isoforms of voltage gated sodium channels are discussed in Clutterbuck et al (2009), J. Med. Chem. 52, 2694-2707. Clutterbuck et al identify nine different Naᵥ isoforms, and consider the possibility of achieving selective inhibition of various isoforms. Naᵥ1.6 is identified as an isoform which is likely to be relevant to neuroprotective activity. Naᵥ1.6 has also been shown to be up-regulated within acute multiple sclerosis lesions (Waxman, S. G. (2006), Nat. Rev. Neurosci., 7, 932-941) and to be associated with the loading of demyelinated axons with damaging levels of calcium (Waxman, S. G. (2008) Nat Clin. Pract. Neurol., 4, 159-169) indicating its potential as a drug target for neuroprotection.

WO 01/057024 discloses a new class of neuroprotective voltage-dependent sodium channel modulators. The compounds disclosed in WO 01/057024 are indazole derivatives which carry a hydrogen atom or an alkyl, aryl or aralkyl group at the 1-position of the indazole moiety.

It has now been found that the indazole derivatives of the formula 1 can inhibit voltage dependent sodium channels. They will therefore be therapeutically useful.

The present invention therefore provides an indazole derivative of formula 1, or a pharmaceutically acceptable salt or N-oxide thereof,

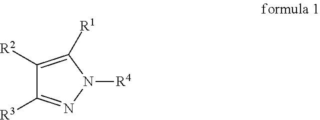

wherein:
either (a) R¹ and R² together represent —(CR′)₄—, or an n-butylene or n-butenylene group, which group is unsubstituted or substituted by one or more R″ substituents, and R³ represents a moiety -A-L-Het-Y-R⁵ or -A-L-Het-H, or (b) R² and R³ together represent —(CR′)₄— or an n-butylene or n-butenylene group, which group is unsubstituted or substituted by one or more R′ substituents, and R¹ represents a moiety -A-L-Het-Y-R⁵ or -A-L-Het-H, wherein each R′ is the same or different and represents hydrogen, halogen, C₁-C₆ alkyl, C₁-C₆ alkoxy, C₁-C₆ haloalkyl, C₁-C₆ haloalkoxy, nitro or cyano, and R″ is the same or different and represents halogen, C₁-C₆ alkyl, C₁-C₆ alkoxy, C₁-C₆ haloalkyl, C₁-C₆ haloalkoxy, nitro or cyano; and
R⁴ represents -L′-A′,
   wherein:
A and A′ are the same or different and each represent a 5- to 10-membered heteroaryl group or a 5- to 10-membered heterocyclyl group, which group is unsubstituted or substituted by one or more substituents selected from halogen, C₁-C₄ alkyl, C₁-C₄ alkoxy, C₁-C₄ haloalkyl, C₁-C₄ haloalkoxy, nitro and cyano substituents;
L and L′ are the same or different and each represent C₁-C₄ alkylene, C₂-C₄ alkenylene or C₂-C₄ alkynylene;
Het is —NR′″—, —O—, or —S—, wherein R′″ is hydrogen or C₁-C₄ alkyl;
Y is —CO—, —CO—O—, —CO—NR_{′}—, or —SO₂—, wherein R_{′} is hydrogen or C₁-C₄ alkyl;
R⁵ is C₁-C₆ alkyl, or is a moiety which is a phenyl ring which is optionally fused to a further phenyl ring or to a 5- or 6-membered heteroaryl or heterocyclyl ring, which moiety is unsubstituted or substituted by one or more substituents selected from halogen, C₁-C₆ alkyl, C₁-C₆ alkoxy, C₁-C₆ haloalkyl, C₁-C₆ haloalkoxy, nitro, cyano, —NR_{′}—CO—R₄₁ , —CO—R_{″}, —O—CO—R_{″}, —CO—O—R_{′} and —CO—N(R_{′})₂ substituents, wherein each R_{′} is the same or different and represents hydrogen or C₁-C₄ alkyl, and each R_{″} is the same or different and represents C₁-C₄ alkyl.

As used herein, a said C₁-C₄ alkylene group or moiety is a linear or branched alkylene group or moiety. Typically, a C₁-C₄ alkylene group or moiety is a C₁-C₂ alkylene group or moiety. Examples of a C₁-C₄ alkylene group or moiety include methylene, ethylene, n-propylene and n-butylene groups and moieties.

As used herein, a C₂-C₄ alkenylene group or moiety is a linear or branched alkenylene group or moiety. Examples include —CH═CH—, —CH₂—CH═CH—, —CH═CH—CH₂—, —CH═CH—CH₂—CH₂—, CH₂—CH═CH—CH₂—, —CH₂—CH₂—CH═CH—, and —CH═CH—CH═CH—.

As used herein, a C₂-C₄ alkynylene group or moiety is a linear or branched alkylene group or moiety. Examples include —C≡C—, —CH₂—C≡C—, —C≡C—CH₂—, —C≡C—CH₂—CH²—, CH₂—C≡C—CH₂—, —CH₂—CH₂—C≡C—, and —C≡C—C≡C—.

As used herein, a halogen is typically chlorine, fluorine, bromine or iodine and is preferably chlorine, bromine or fluorine.

As used herein, a C₁-C₆ alkyl group or moiety is a linear or branched alkyl group or moiety containing from 1 to 6 carbon atoms. Typically a C₁-C₆ alkyl group or moiety is a C₁-C₄ alkyl group or moiety. Examples of C₁-C₆ alkyl groups and moieties include methyl, ethyl, n-propyl, i-propyl, n-butyl, i-butyl, t-butyl and 3-methyl-butyl. For the avoidance of doubt, where two alkyl moieties are present in a group, the alkyl moieties may be the same or different.

As used herein, a C₁-C₆ alkoxy group is typically a said C₁-C₆ alkyl group attached to an oxygen atom. A haloalkyl or haloalkoxy group is typically a said alkyl or alkoxy group substituted by one or more said halogen atoms. Typically, it is substituted by 1, 2 or 3 said halogen atoms. Preferred haloalkyl and haloalkoxy groups include perhaloalkyl and perhaloalkoxy groups such as —CX₃ and —OCX₃ wherein X is a said halogen atom, for example chlorine and fluorine. Particularly preferred haloalkyl groups are —CF₃ and —CCl₃. Particularly preferred haloalkoxy groups are —OCF₃ and —OCCl₃.

As used herein, a 5- to 10-membered heteroaryl group or moiety is typically a monocyclic 5- to 10-membered aromatic group or moiety, such as a 5- or 6-membered ring containing at least one heteroatom, for example 1, 2 or 3 heteroatoms, selected from O, S and N. Examples include pyridyl, pyrazinyl, pyrimidinyl, pyridazinyl, furanyl, thienyl, pyrazolidinyl, pyrrolyl, oxadiazolyl, oxazolyl, isoxazolyl, thiazolyl, thiadiazolyl, imidazolyl, pyridazolyl, pyrazolyl, groups. Preferred examples include pyrrolyl, oxadiazolyl, thiazolyl, pyridyl, imidazolyl and thienyl groups. Oxadiazolyl and imidazolyl groups are further preferred.

As used herein, a 5- to 10-membered heterocyclyl group or moiety is typically a monocyclic non-aromatic, saturated or unsaturated C₅-C₁₀ carbocyclic ring in which one or more, for example 1, 2 or 3, of the carbon atoms are replaced with a moiety selected from N, O, S, S(O) and S(O)₂. Typically, it is a 5- to 6-membered ring. Suitable heterocyclyl groups and moieties include pyrazolidinyl, piperidyl, piperazinyl, thiomorpholinyl, S-oxo-thiomorpholinyl, S,S-dioxo-thiomorpholinyl, morpholinyl, pyrrolidinyl, pyrrolinyl, imidazolidinyl, imidazolinyl, 1,3-dioxolanyl, 1,4-dioxolyl and pyrazolinyl groups and moieties. A 1,3-dioxolanyl group or moiety is preferred.

In one embodiment, the invention provides an indazole derivative of formula 1, or a pharmaceutically acceptable salt or N-oxide thereof, wherein:
either (a) R¹ and R² together represent —(CR′)₄—, and R³ represents a moiety -A-L-Het-Y-R⁵ or -A-L-Het-H, or (b) R² and R³ together represent —(CR′)₄—, and R¹ represents a moiety -A-L-Het-Y-R⁵ or -A-L-Het-H, wherein each R′ is the same or different and is as defined above; and
R⁴, A, L, Het, Y and R⁵ are as defined above.

In this embodiment, the compound of formula 1 may be a compound of formula (1a) or (1b),

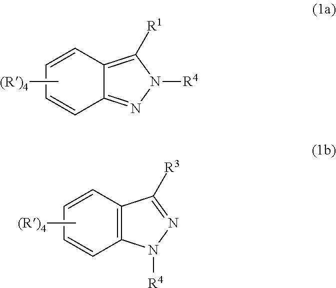

wherein R¹ and R³ represent a moiety -A-L-Het-Y-R⁵ or -A-L-Het-H, and R′, R⁴, A, L, Het, Y and R⁵ are as defined above.

Preferably, each R′ is the same or different and represents hydrogen, halogen, C₁-C₆ alkyl, C₁-C₆ alkoxy, C₁-C₆ haloalkyl or C₁-C₆ haloalkoxy. More preferably, each R′ is the same or different and represents hydrogen, halogen or C₁-C₄ alkoxy. Most preferably, each R′ is the same or different and represents hydrogen, fluorine, chlorine, bromine or methoxy.

Preferably, 2, 3 or 4 of the R′ groups are hydrogen. More preferably, when two of the R′ groups are hydrogen atoms, the other two le groups are halogen atoms, preferably chlorine atoms.

Typically, A is unsubstituted or substituted by 1 or 2 substituents selected from halogen, C₁-C₄ alkyl, C₁-C₄ alkoxy and C₁-C₄ haloalkyl substituents. Preferably, A is unsubstituted.

Typically, A is a 5 to 6 membered heteroaryl or 5 to 6 membered heterocyclyl group. More typically, A is a 5 to 6 membered heteroaryl group. Preferably, A is other than furanyl or pyrrolyl. More preferably, A is pyridyl, pyrazinyl, pyrimidinyl, pyridazinyl, thienyl, pyrazolidinyl, oxadiazolyl, oxazolyl, isoxazolyl, thiazolyl, thiadiazolyl, imidazolyl, pyridazolyl or pyrazolyl. Still more preferably A is an oxadiazolyl group. Most preferably A is a 1,2,4-oxadiazolyl group.

Typically, L is C₁-C₄ alkylene. Preferably, L is C₁-C₂ alkylene. More preferably L is methylene.

Typically, Het is —NR′″—. Preferably, Het is —NH—. Preferably, -Het-H is —NH₂.

For the avoidance of doubt, the left hand side of the depicted Y moieties is attached to the Het moiety, and the right hand side of the depicted Y moieties is attached to the R⁵ moiety. Typically, Y is —CO—, —CO—O— or —S(O)₂—. Preferably, the moiety -Het-Y- represents —NH—CO—, —NH—CO—O—, or —NH—S(O)₂—.

In the embodiment where R⁵ is C₁-C₆ alkyl, R⁵ is preferably t-butyl.

In the embodiment where R⁵ is a moiety which is a phenyl ring which is optionally fused to a further phenyl ring or to a 5- or 6-membered heteroaryl or heterocyclyl ring, said moiety is typically unsubstituted or substituted by 1, 2 or 3 substituents. Typically, these substituents are selected from halogen, C₁-C₄ alkyl, C₁-C₄ alkoxy, C₁-C₂ haloalkyl, C₁-C₂haloalkoxy, nitro and —NR_{′}—CO—R_{″} substituents, wherein R_{′} is a hydrogen atom and R_{″} is C₁-C₄ alkyl. More preferably, the substituents are selected from methyl, methoxy, t-butyl, trifluoromethyl, trifluoromethoxy, fluoro, chloro, nitro and —NH—CO—CH₃ substituents. Preferably, the moiety which is a phenyl ring optionally fused to a further phenyl ring or to a 5- or 6-membered heteroaryl or heterocyclyl ring is phenyl, naphthyl, indolyl, or benzodioxolyl. Most preferably the moiety is phenyl.

Typically the moiety -A-L-Het-H is other than a 2-(hydroxymethyl)furan-5-yl or N-methyl-2-(hydroxymethyl)-pyrrol-5-yl group. In this embodiment, preferably the moiety -A-L-Het-H is other than a (hydroxymethyl)furanyl or N-methyl(hydroxymethyl)pyrrolyl group. More preferably the moiety -A-L-Het-H is other than -A,-L-Het-H, wherein A, is furanyl or pyrrolyl.

L′ is preferably C₁-C₄ alkylene. More preferably, L′ is C₁-C₂ alkylene. Typically, L′ is methylene or ethlyene.

Typically, A′ is unsubstituted or substituted by 1, 2 or 3 substituents selected from halogen, C₁-C₄ alkyl, C₁-C₄ alkoxy, C₁-C₄ haloalkyl and C₁-C₄ haloalkoxy substituents. Preferably the substituents are selected from C₁-C₄ alkyl groups, more preferably C₁-C₂ alkyl groups, most preferably methyl groups.

More preferably, A′ is unsubstituted or substituted by one substituent. Most preferably A′ is unsubstituted or substituted by one methyl substituent.

A′ is typically a 5- to 6-membered heteroaryl or 5- to 6-membered heterocyclyl group, preferably a 5- to 6-membered heteroaryl group. More preferably A′ is thiazolyl, pyridyl, imidazolyl or thienyl. Most preferably A′ is imidazolyl.

Preferably, -L′-A′ is —CH₂-A″, wherein A″ is thiazolyl or pyridyl, or -L′-A′ is —CH₂—CH₂-A′″, wherein A′″ is imidazolyl or thienyl.

Preferred compounds of the invention are indazole derivatives of formula (1a) or (1b), pharmaceutically acceptable salts thereof and N-oxides thereof wherein:
R¹ and R³ represent a moiety -A-L-Het-Y-R⁵ or -A-L-Het-H;
each R′ is the same or different and represents hydrogen, halogen, C₁-C₆ alkyl, C₁-C₆ alkoxy, C₁-C₆ haloalkyl or C₁-C₆ haloalkoxy, provided that 2, 3 or 4 of the R′ groups are hydrogen;
A is a 5 to 6 membered heteroaryl or 5 to 6 membered heterocyclyl group, which group is unsubstituted or substituted by 1 or 2 substituents selected from halogen, C₁-C₄ alkyl, C₁-C₄ alkoxy and C₁-C₄ haloalkyl substituents;
L is C₁-C₄ alkylene;
Het is —NR′″—, wherein R′″ is hydrogen or C₁-C₄ alkyl;
Y is —CO—, —CO—O— or —S(O)₂—;
R⁵ is t-butyl or a phenyl, naphthyl, indolyl or benzodioxolyl group, which group is unsubstituted or substituted by 1, 2 or 3 substituents selected from halogen, C₁-C₄ alkyl, C₁-C₄ alkoxy, C₁-C₂ haloalkyl, C₁-C₂ haloalkoxy, nitro and —NR_{″}—CO—R_{″} substituents, wherein R_{′} is a hydrogen atom and R_{″} is C₁-C₄ alkyl; and
R⁴ represents -L′-A′, wherein L′ is C₁-C₄ alkylene and A′ is a 5 to 6 membered heteroaryl group or 5- to 6-membered heterocyclyl group, which group is unsubstituted or substituted by 1, 2 or 3 substituents selected from halogen, C₁-C₄ alkyl, C₁-C₄ alkoxy, C₁-C₄ haloalkyl and C₁-C₄ haloalkoxy substituents.

Particularly preferred compounds of the invention are indazole derivatives of formula (1a) or (1b), pharmaceutically acceptable salts thereof and N-oxides thereof wherein:
R¹ and R³ represent a moiety -A-L-Het-Y-R⁵ or -A-L-Het-H;
each R′ is the same or different and represents hydrogen, halogen, or C₁-C₄ alkoxy, provided that 2, 3 or 4 of the R′ groups are hydrogen;
A is an unsubstituted 5- to 6-membered heteroaryl group;
L is C₁-C₂ alkylene;
Het is —NH—;
Y is —CO—, —CO—O— or —S(O)₂—;
R⁵ is t-butyl or a phenyl, naphthyl, indolyl or benzodioxolyl group, which group is unsubstituted or substituted by 1, 2 or 3 substituents selected from methyl, methoxy, t-butyl, trifluoromethyl, trifluoromethoxy, fluoro, chloro, nitro and —NH—CO—CH₃ substituents; and
R⁴ represents -L′-A′, wherein L′ is C₁-C₂ alkylene and A′ is a 5 to 6 membered heteroaryl group, which group is unsubstituted or substituted by 1, 2 or 3 C₁-C₄ alkyl substituents.

Most preferred compounds of the invention are indazole derivatives of formula (1a) or (1b), pharmaceutically acceptable salts thereof and N-oxides thereof wherein:
R¹ and R³ represent a moiety -A-L-Het-Y-R⁵ or -A-L-Het-H;
each R′ is the same or different and represents hydrogen, fluorine, chlorine, bromine or methoxy, provided that 2, 3 or 4 of the R′ groups are hydrogen and, when two of the R′ groups are hydrogen atoms, the other two R′ groups are chlorine atoms;
A is a 1,2,4-oxadiazolyl group;
L is methylene;
Het is —NH—;
Y is —CO—, —CO—O— or —S(O)₂—;
R⁵ is t-butyl or a phenyl, naphthyl, indolyl or benzodioxolyl group, which group is unsubstituted or substituted by 1, 2 or 3 substituents selected from methyl, methoxy, t-butyl, trifluoromethyl, trifluoromethoxy, fluoro, chloro, nitro and —NH—CO—CH₃ substituents; and
R⁴ represents -L′-A′, wherein L′ is methylene or ethlyene and A′ is a thiazolyl, pyridyl, imidazolyl or thienyl group, which group is unsubstituted or substituted by one methyl substituent.

Preferred compounds of formula 1 include:
1. tert-butyl (5-(1-(2-(1H-imidazol-1-yl)ethyl)-1H-indazol-3-yl)-1,2,4-oxadiazol-3-yl)methylcarbamate
2. (5-(1-(2-(1H-imidazol-1-yl)ethyl)-1H-indazol-3-yl)-1,2,4-oxadiazol-3-yl)methanamine
3. tert-butyl (5-(1-(2-(1H-imidazol-1-yl)ethyl)-1H-indazol-3-yl)-1,2,4-oxadiazol-3-yl)methylcarbamate
4. tert-butyl (5-(2-(2-(1H-imidazol-1-yl)ethyl)-2H-indazol-3-yl)-1,2,4-oxadiazol-3-yl)methylcarbamate
5. (5-(2-(2-(1H-imidazol-1-yl)ethyl)-2H-indazol-3-yl)-1,2,4-oxadiazol-3-yl)methanamine
6. N-((5-(2-(2-(1H-imidazol-1-yl)ethyl)-2H-indazol-3-yl)-1,2,4-oxadiazol-3-yl)methyl)benzamide
7. N-((5-(2-(2-(1H-imidazol-1-yl)ethyl)-2H-indazol-3-yl)-1,2,4-oxadiazol-3-yl)methyl)-2,3-dimethoxybenzamide
8. N-((5-(2-(2-(1H-imidazol-1-yl)ethyl)-2H-indazol-3-yl)-1,2,4-oxadiazol-3-yl)methyl-2-methylbenzamide
9. N-((5-(2-(2-(1H-imidazol-1-yl)ethyl)-2H-indazol-3-yl)-1,2,4-oxadiazol-3-yl)methyl)-4-acetamidobenzamide
10. N-((5-(2-(2-(1H-imidazol-1-yl)ethyl)-2H-indazol-3-yl)-1,2,4-oxadiazol-3-yl)methyl)-4-chlorobenzamide
11. N-((5-(2-(2-(1H-imidazol-1-yl)ethyl)-2H-indazol-3-yl)-1,2,4-oxadiazol-3-yl)methyl)-2,3-difluorobenzamide
12. N-((5-(2-(2-(1H-imidazol-1-yl)ethyl)-2H-indazol-3-yl)-1,2,4-oxadiazol-3-yl)methyl)-2-naphthamide
13. N-((5-(2-(2-(1H-imidazol-1-yl)ethyl)-2H-indazol-3-yl)-1,2,4-oxadiazol-3-yl)methyl)-3-methyl-4-nitrobenzamide
14. N-((5-(2-(2-(1H-imidazol-1-yl)ethyl)-2H-indazol-3-yl)-1,2,4-oxadiazol-3-yl)methyl)benzo[d][1,3]dioxole-5-carboxamide
15. N-((5-(2-(2-(1H-imidazol-1-yl)ethyl)-2H-indazol-3-yl)-1,2,4-oxadiazol-3-yl)methyl)-3-methylbenzamide
16. N-((5-(2-(2-(1H-imidazol-1-yl)ethyl)-2H-indazol-3-yl)-1,2,4-oxadiazol-3-yl)methyl)-3-(trifluoromethoxy)benzamide
17. N-((5-(2-(2-(1H-imidazol-1-yl)ethyl)-2H-indazol-3-yl)-1,2,4-oxadiazol-3-yl)methyl)benzenesulfonamide
18. N-(4-(N-((5-(2-(2-(1H-imidazol-1-yl)ethyl)-2H-indazol-3-yl)-1,2,4-oxadiazol-3-yl)methyl)sulfamoyl)phenyl)acetamide
19. N-((5-(1-(2-(1H-imidazol-1-yl)ethyl)-1H-indazol-3-yl)-1,2,4-oxadiazol-3-yl)methyl)benzamide
20. N-((5-(1-(2-(1H-imidazol-1-yl)ethyl)-1H-indazol-3-yl)-1,2,4-oxadiazol-3-yl)methyl)-2,3,4-trimethoxybenzamide
21. N-((5-(1-(2-(1H-imidazol-1-yl)ethyl)-1H-indazol-3-yl)-1,2,4-oxadiazol-3-yl)methyl)-4-chlorobenzenesulfonamide
22. N-((5-(1-(2-(1H-imidazol-1-yl)ethyl)-1H-indazol-3-yl)-1,2,4-oxadiazol-3-yl)methyl)-2,3-dimethoxybenzenesulfonamide
23. N-((5-(1-(2-(1H-imidazol-1-yl)ethyl)-1H-indazol-3-yl)-1,2,4-oxadiazol-3-yl)methyl)-1H-indole-4-carboxamide
24. N-((5-(1-(2-(1H-imidazol-1-yl)ethyl)-1H-indazol-3-yl)-1,2,4-oxadiazol-3-yl)methyl)-4-(trifluoromethoxy)benzenesulfonamide
25. N-((5-(1-(2-(1H-imidazol-1-yl)ethyl)-1H-indazol-3-yl)-1,2,4-oxadiazol-3-yl)methyl)-2-methylbenzenesulfonamide
26. N-((5-(1-(2-(1H-imidazol-1-yl)ethyl)-1H-indazol-3-yl)-1,2,4-oxadiazol-3-yl)methyl)-3-(trifluoromethyl)benzenesulfonamide
27. N-((5-(1-(2-(1H-imidazol-1-yl)ethyl)-1H-indazol-3-yl)-1,2,4-oxadiazol-3-yl)methyl)-4-tert-butylbenzenesulfonamide
28. N-((5-(1-(2-(1H-imidazol-1-yl)ethyl)-1H-indazol-3-yl)-1,2,4-oxadiazol-3-yl)methyl)-4-tert-butylbenzamide
29. N-((5-(1-(2-(1H-imidazol-1-yl)ethyl)-1H-indazol-3-yl)-1,2,4-oxadiazol-3-yl)methyl)-2,3-dimethoxybenzamide
30. N-((5-(1-(2-(1H-imidazol-1-yl)ethyl)-1H-indazol-3-yl)-1,2,4-oxadiazol-3-yl)methyl)-3-(trifluoromethoxy)benzamide
31. tert-butyl (5-(1-(2-methylthiazol-4-yl)methyl)-1H-indazol-3-yl)-1,2,4-oxadiazol-3-yl)methylcarbamate
32. (5-(1-(2-methylthiazol-4-yl)methyl)-1H-indazol-3-yl)-1,2,4-oxadiazol-3-yl)methanamine
33. tert-butyl (5-(2-(2-(thiophen-2-yl)ethyl)-2H-indazol-3-yl)-1,2,4-oxadiazol-3-yl)methylcarbamate
34. N-((5-(1((2-methylthiazol-4-yl)methyl)-1H-indazol-3-yl)-1,2,4-oxadiazol-3-yl)methyl)benzamide
35. (5-(2-(2-(thiophen-2-yl)ethyl)-2H-indazol-3-yl)-1,2,4-oxadiazol-3-yl)methanamine
36. N-((5-(2-(2-(thiophen-2-yl)ethyl)-2H-indazol-3-yl)-1,2,4-oxadiazol-3-yl)methyl)benzamide
37. tert-butyl (5-(2-((pyridin-2-yl)methyl)-2H-indazol-3-yl)-1,2,4-oxadiazol-3-yl)methylcarbamate
38. (5-(2-((pyridin-2-yl)methyl)-2H-indazol-3-yl)-1,2,4-oxadiazol-3-yl)methanamine
39. N-((5-(2-((pyridin-2-yl)methyl)-2H-indazol-3-yl)-1,2,4-oxadiazol-3-yl)methyl)benzamide
40. N-((5-(2-(2-(1H-imidazol-1-yl)ethyl)-2H-indazol-3-yl)-1,2,4-oxadiazol-3-yl)methyl)-2-methoxybenzamide
41. N-((5-(2-(2-(1H-imidazol-1-yl)ethyl)-2H-indazol-3-yl)-1,2,4-oxadiazol-3-yl)methyl)-3-methoxybenzamide
42. N-((5-(2-(2-(1H-imidazol-1-yl)ethyl)-2H-indazol-3-yl)-1,2,4-oxadiazol-3-yl)methyl)-4-methoxybenzamide
43. N-((5-(2-(2-(1H-imidazol-1-yl)ethyl)-2H-indazol-3-yl)-1,2,4-oxadiazol-3-yl)methyl)-4-(trifluoromethoxy)benzamide
44. (5-(2-(2-(1H-imidazol-1-yl)ethyl)-5-bromo-2H-indazol-3-yl)-1,2,4-oxadiazol-3-yl)methanamine
45. N-((5-(2-(2-(1H-imidazol-1-yl)ethyl)-5-bromo-2H-indazol-3-yl)-1,2,4-oxadiazol-3-yl)methyl)-2,3-dimethoxybenzamide
46. N-((5-(2-(2-(1H-imidazol-1-yl)ethyl)-5-bromo-2H-indazol-3-yl)-1,2,4-oxadiazol-3-yl)methyl)-3-(trifluoromethoxy)benzamide
47. (5-(2-(2-(1H-imidazol-1-yl)ethyl)-5-fluoro-2H-indazol-3-yl)-1,2,4-oxadiazol-3-yl)methanamine
48. (5-(2-(2-(1H-imidazol-1-yl)ethyl)-4,6-dichloro-2H-indazol-3-yl)-1,2,4-oxadiazol-3-yl)methanamine
49. N-((5-(2-(2-(1H-imidazol-1-yl)ethyl)-4,6-dichloro-2H-indazol-3-yl)-1,2,4-oxadiazol-3-yl)methyl)-2,3-dimethoxybenzamide
50. N-((5-(2-(2-(1H-imidazol-1-yl)ethyl)-5-fluoro-2H-indazol-3-yl)-1,2,4-oxadiazol-3-yl)methyl)-2,3-dimethoxybenzamide pharmaceutically acceptable salt thereof, N-oxides thereof and mixtures thereof.

The compounds of the invention can be prepared by standard techniques familiar to those of skill in the art. For example, compounds of formula 1, wherein R¹, R², R³ and R⁴ are as defined above, can be prepared by following Scheme 1 or 2 as set out below,

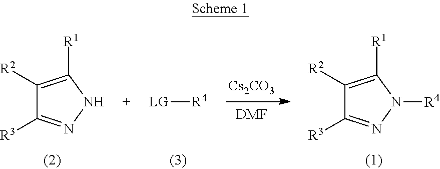

wherein LG is a leaving group, for example a halogen, mesylate or triflate. Preferably, LG is a bromide group.

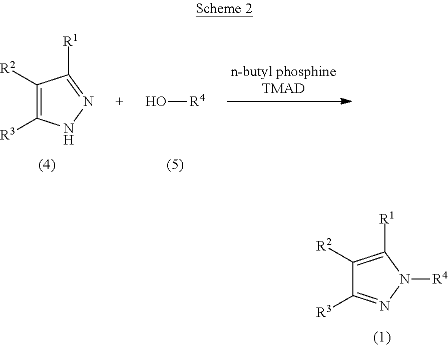

The starting materials of formulae (2) and (4) are known compounds or may be prepared by analogy with known methods. For example, compounds of formula (10) in which R represents a moiety -A-L-Het-Y-R⁵ or -A-L-Het-H as defined above, can be prepared according to Scheme 3 below. These compounds correspond to compounds of formulae (2) and (4) in which R¹ and R², or R² and R³ together represent —(CR′)₄— and R³ or R′ respectively represent a moiety -A-L-Het-Y-R⁵ or -A-L-Het-H as defined above.

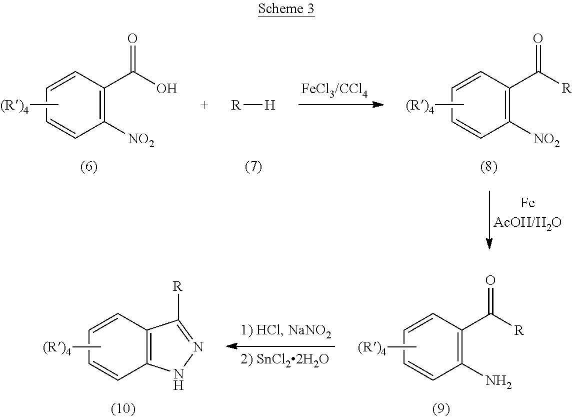

Compounds of formula (13) in which R represents a moiety -A-L-Het-Y-R⁵ or -A-L-Het-H as defined above, can be prepared according to Scheme 4 below. These compounds correspond to compounds of formulae (2) and (4) in which R¹ and R², or R² and R³ together represent an n-butylene group and R³ or R¹ respectively represent a moiety -A-L-Het-Y-R⁵ or -A-L-Het-H as defined above.

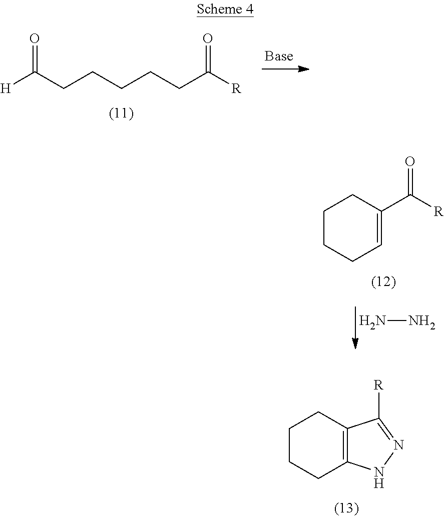

Starting materials of formula (2) or (4) wherein R¹ and R² or R² and R³ together represent a substituted n-butylene group, or a substituted or unsubstituted n-butenylene group, and R³ or R¹ respectively represent a moiety -A-L-Het-Y-R⁵ or -A-L-Het-H as defined above, can be prepared by analogy with scheme 4, using a starting molecule with the appropriate substitution and/or unsaturation.

Compounds of formula (1a) where A is 1,2,4-oxadiazolyl, L is methylene and -Het-H is —NH₂ or -Het-Y-R⁵ is —NH—CO—O-(t-butyl), —NH—CO-(phenyl) or —NH—S(O)₂-(phenyl), and R⁴ is as defined above, can be prepared by following general scheme 5 as set out below.

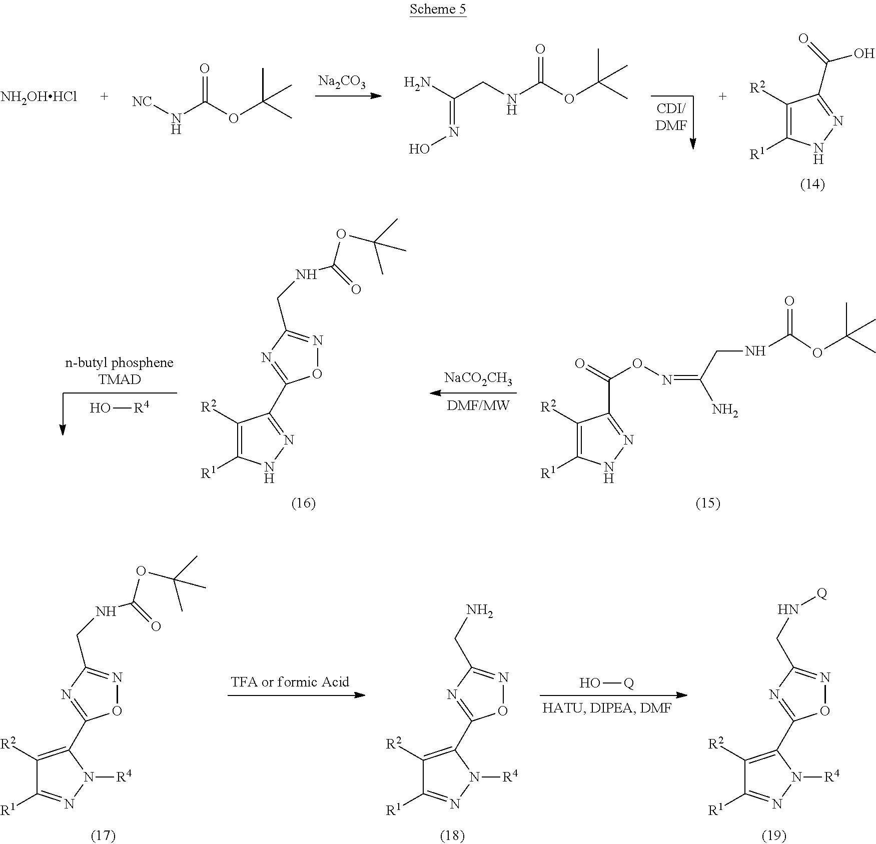

In scheme 5, compound 18, which is a compound of formula (1a) where -Het-H is —NH₂, may be converted to compound 19, where -Het-Y-R⁵ is —NH—CO—O-(t-butyl) or —NH—CO-(phenyl) by treatment with a compound HO-Q, wherein Q is a moiety —CO—O-(t-butyl) or —CO-(phenyl). The coupling partner is a carboxylic acid, and the HATU generates an activated ester.

Compounds of formula (1b) where A is 1,2,4-oxadiazolyl, L is methylene and -Het-H is —NH₂ or -Het-Y-R⁵ is —NH—CO—O-(t-butyl) or —NH—CO-(phenyl), and R⁴ is as defined above, can be prepared according to scheme 6 as set out below,

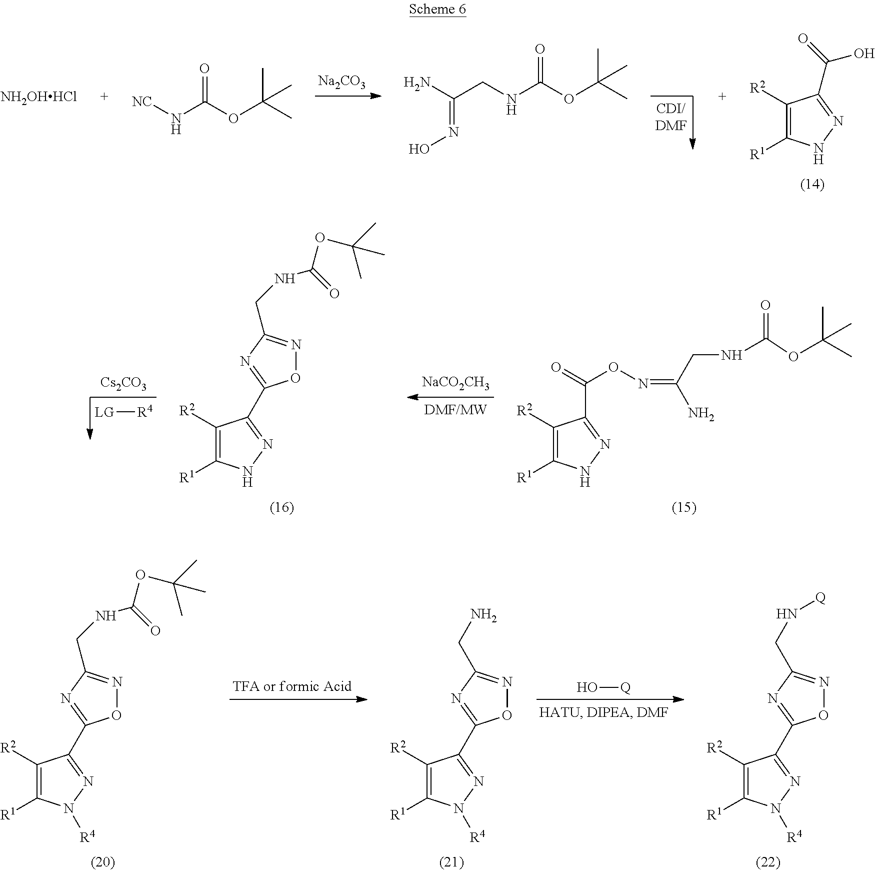

wherein Q is as defined above.

Compounds of formula 1 containing one or more chiral centre may be used in enantiomerically or diastereoisomerically pure form, or in the form of a mixture of isomers. For the avoidance of doubt, the compounds of formula 1 can, if desired, be used in the form of solvates. Further, for the avoidance of doubt, the compounds of the invention may be used in any tautomeric form.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

A compound of formula 1 can be salified by known methods, by contacting the compound with an appropriate acid or base.

The compounds of the invention do not inhibit cytochrome P450 to a significant extent. Cytochrome P450 (CYP 450) enzymes are responsible for the oxidative metabolism of drugs in animals. The inhibition of this enzyme is undesirable because it could lead to the accumulation of other drugs to toxic levels in the body.

Certain compounds of the invention are found to be selective Naᵥ1.6 inhibitors. These compounds are highly preferred. They are indazole derivatives of formula (1c), pharmaceutically acceptable salts and N-oxides thereof,

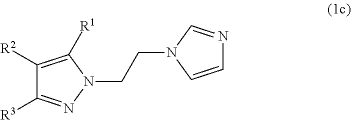

wherein:
either (a) R₁ and R₂ are linked to form a group —(CH)₄— and R₃ is a moiety W, or (b) R₂ and R₃ are linked to form a group —(CH)₄— and R₁ is a moiety W; and
the moiety W is a moiety,

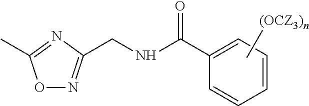

wherein Z is a hydrogen atom or a fluorine atom, and n is 1, 2, or 3.

The compounds of the invention are capable of inhibiting voltage dependent sodium channels. They can therefore be used, for example, to protect cells against damage which results from overstimulation of sodium channels. Nitric oxide (NO) has recently been implicated in overstimulation of voltage dependent sodium channels. The compounds of the invention can therefore be used to protect cells against NO mediated damage resulting from the overstimulation of voltage dependent sodium channels.

The compounds of the invention are, in particular, effective in protecting neuronal white matter, or myelin coated nerve cell fibres. They therefore have a neuroprotective effect on ganglion cells and axons and can be used in the treatment or prevention of an affective disorder, an anxiety disorder, a behavioural disorder, a cardiovascular disorder, a central or peripheral nervous system degenerative disorder, a central nervous system injury, a cerebral ischaemia, a chemical injury or substance abuse disorder, a cognitive disorder, an eating disorder, an eye disease, Parkinson's disease, pain or a seizure disorder.

Examples of affective disorders which can be treated or prevented with the compounds of the invention include mood disorders, bipolar disorders (both Type 1 and Type II) such as seasonal affective disorder, depression, manic depression, atypical depression and monodepressive disease, schizophrenia, psychotic disorders, mania and paranoia.

Examples of anxiety disorders which can be treated or prevented with the compounds of the invention include generalised anxiety disorder (GAD), panic disorder, panic disorder with agoraphobia, simple (specific) phobias (e.g. arachnophobia, performance anxiety such as public speaking), social phobias, post-traumatic stress disorder, anxiety associated with depression, and obsessive compulsive disorder (OCD).

Examples of behavioural disorders which can be treated or prevented with the compounds of the invention include behavioural and psychological signs and symptoms of dementia, age-related behavioural disorders, pervasive development disorders such as autism, Aspergers Syndrome, Retts syndrome and disintegrative disorder, attention deficit disorder, aggressivity, impulse control disorders and personality disorder.

Examples of cardiovascular disorders which can be treated or prevented with the compounds of the invention include atherosclerosis, cardiac arrest, thrombosis, complications arising from coronary artery bypass surgery, myocardial infarction, reperfusion injury, intermittant claudication, ischaemic retinopathy, angina, pre-eclampsia, hypertension, congestive cardiac failure, restenosis following angioplasty, sepsis and septic shock.

Examples of central and peripheral nervous system degenerative disorders which can be treated or prevented with the compounds of the invention include corticobasal degeneration, demyelinating disease such as multiple sclerosis and disseminated sclerosis, Freidrich's ataxia, motorneurone diseases such as amyotrophic lateral sclerosis and progressive bulbar atrophy, multiple system atrophy, myelopathy, radiculopathy, peripheral neuropathies such as diabetic neuropathy, tabes dorsalis, drug-induced neuropathy and vitamin deficiency, systemic lupus erythamatosis, granulomatous disease, olivo-ponto-cerebellar atrophy, progressive pallidal atrophy, progressive supranuclear palsy and spasticity.

Examples of central nervous system injuries which can be treated with the compounds of the invention include traumatic brain injury, neurosurgery (surgical trauma), neuroprotection for head injuries, raised intracranial pressure, cerebral oedema, hydrocephalus and spinal cord injury.

Examples of cerebral ischaemias which can be treated or prevented with the compounds of the invention include transient ischaemic attack, stroke, for example thrombotic stroke, ischaemic stroke, embolic stroke, haemorrhagic stroke or lacunar stroke, subarachnoid haemorrhage, cerebral vasospasm, peri-natal asphyxia, drowning, cardiac arrest and subdural haematoma.

Examples of chemical injuries and substance abuse disorders which can be treated or prevented with the compounds of the invention include drug dependence, for example opiate dependence, benzodiazepine addition, amphetamine addiction and cocaine addiction, alcohol dependence, methanol toxicity, carbon monoxide poisoning and butane inhalation.

Examples of cognitive disorders which can be treated or prevented with the compounds of the invention include dementia, Alzheimers Disease, Frontotemporal dementia, multi-infarct dementia, AIDS dementia, dementia associated with Huntingtons Disease, Lewy body Dementia, Senile dementia, age-related memory impairment, cognitive impairment associated with dementia, Korsakoff syndrome and dementia pugilans.

Examples of eating disorders which can be treated or prevented with the compounds of the invention include anorexia nervosa, bulimia, Prader-Willi syndrome and obesity.

Examples of eye diseases which can be treated or prevented with the compounds of the invention include drug-induced optic neuritis, cataract, diabetic neuropathy, ischaemic retinopathy, retinal haemorrhage, retinitis pigmentosa, acute glaucoma, in particular acute normal tension glaucoma, chronic glaucoma, in particular chronic normal tension glaucoma, macular degeneration, retinal artery occlusion and retinitis.

Examples of Parkinson's diseases which can be treated or prevented with the compounds of the invention include drug-induced Parkinsonism, post-encephalitic Parkinsonism, Parkinsonism induced by poisoning (for example MPTP, manganese or carbon monoxide poisoning), Dopa-responsive dystonia-Parkinsonism, post-traumatic Parkinson's disease (punch-drunk syndrome), Parkinson's with on-off syndrome, Parkinson's with freezing (end of dose deterioration) and Parkinson's with prominent dyskinesias.

Examples of pains which can be treated or prevented with the compounds of the invention include acute pain (e.g. musculoskeletal and post-operative pain) and chronic pain such as osteoarthritis, neuropathic pain, cancer pain, trigeminal neuralgia, migraine and other conditions associated with cephalic pain, primary and secondary hyperalgesia, inflammatory pain, nociceptive pain, tabes dorsalis, phantom limb pain, spinal cord injury pain, central pain, post-herpetic pain and HIV pain, non-cardiac chest pain, irritable bowel syndrome and pain associated with bowel disorders and dyspepsia.

Examples of seizure disorders which can be treated or prevented with the compounds of the invention include epilepsy and post-traumatic epilepsy, partial epilepsy (simple partial seizures, complex partial seizures, and partial seizures secondarily generalised seizures), generalised seizures, including generalised tonic-clonic seizures (grand mal), absence seizures (petit mal), myoclonic seizures, atonic seizures, clonic seizures, and tonic seizures, Lennox Gastaut, West Syndome (infantile spasms), multiresistant seizures and seizure prophylaxis (anti-epileptogenic).

Typically, the compounds of the invention are for use in the treatment or prevention of normal tension glaucoma, multiple sclerosis, a motorneurone disease, stroke, spinal cord injury, Alzheimer's disease, Parkinson's disease or pain.

In a preferred embodiment, the compounds of the invention are for use in the treatment or prevention of an affective disorder, an anxiety disorder, a behavioural disorder, a central or peripheral nervous system degenerative disorder, a central nervous system injury, a cerebral ischaemia, a chemical injury or substance abuse disorder, a cognitive disorder, an eating disorder, an eye disease, Parkinson's disease, pain or a seizure disorder, which disorder is other than stroke.

Preferably, the compounds of the invention are for use in the treatment or prevention of normal tension glaucoma, multiple sclerosis, a motorneurone disease, spinal cord injury, Alzheimer's disease, Parkinson's disease or pain. More preferably the compounds of the invention are for use in the treatment or prevention of multiple sclerosis or pain.

The compounds of the invention are particularly effective in protecting ganglion cells and axons of the optic nerve from damage. They are therefore particularly effective in the treatment or prevention of glaucoma, for example acute glaucoma or chronic glaucoma. As the compounds of the invention are effective as neuroprotectors for white matter, they can be used specifically in the treatment or prevention of normal tension, or normal pressure, glaucoma. Accordingly, when the compounds of the invention are used in the treatment or prevention of glaucoma, they are preferably used in the treatment or prevention of normal tension, or normal pressure, glaucoma.

A further preferred use of the compounds of the invention is in the treatment or prevention of multiple sclerosis.

The compounds of the invention may be administered in a variety of dosage forms. Thus, they can be administered orally, for example as tablets, troches, lozenges, aqueous or oily suspensions, dispersible powders or granules. The compounds of the invention may also be administered parenterally, either subcutaneously, intravenously, intramuscularly, intrasternally, transdermally or by infusion techniques. The compounds may also be administered as suppositories.

A compound of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginte, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

A therapeutically effective amount of a compound of the invention is administered to a patient. A typical daily dose is from about 0.1 to 50 mg per kg of body weight, according to the activity of the specific compound, the age, weight and conditions of the subject to be treated, the type and severity of the disease and the frequency and route of administration. Preferably, daily dosage levels are from 5 mg to 2 g.

The Examples which follow illustrate the invention.

### PRODUCTION EXAMPLE 1

### Synthesis of N-(tert-butoxycarbonyI)-2-aminoacetamidoxime

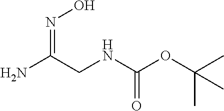

To a solution of Na₂CO₃ (8.48 g, 0.08 mol, 1 equiv.) in water (35 mL) was slowly added a solution of hydroxylamine hydrochloride (5.56 g, 0.08 mol, 1 equiv.) in water (35 mL). The aqueous solution of hydroxylamine was added to a solution of the N-(tert-butoxycarbonyl)-2-aminoacetonitrile (12.5 g, 0.08 mol, 1 equiv.) in methanol (200 mL). The clear light yellow solution was heated at reflux (90° C.) overnight (23 h) with more water (100 mL) being added as necessary to dissolve the precipitate formed. The methanol was removed under reduced pressure, and the residue partitioned between ethyl acetate (100 mL) and water (100 mL). The aqueous phase was extracted with ethyl acetate, and the combined organic phase was dried over magnesium sulfate. The solvent was removed in vacuo to give a white powder. Yield 9.83 g, 65%; m.p. 147-148° C.; δ_{H} (MeOH-d₆) 1.44 (9H, s, O′Bu), 3.64 (2H, s, CH₂); δ_{C} (MeOH-d₆) 158.8, 154.7, 80.7, 41.4, 28.7; LC-MS-EI 190.2 (MH⁺, 100); Found (CI) 190.11879 C₇H₁₅N₃O₃ (MH⁺) requires 190.11916.

### PRODUCTION EXAMPLE 2

### Synthesis of

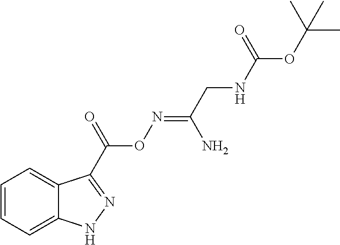

To a round bottomed flask was added indazole-3-carboxylic acid (21.09 g, 0.13 mol, 1 equiv,) in DMF (500 mL) under nitrogen. Carbodiimidazole (CDI) (23.19 g, 0.14 mol, 1.1 equiv.) was added to the resulting slightly yellow solution and this was stirred at room temperature for 30 minutes. N-(tert-butoxycarbonyl)-2-aminoacetamidoxime from production Example 1 (27.06 g, 0.14 mol, 1.1 equiv.) in DMF (100 mL) was then added and the solution was stirred overnight at room temperature. The solvent was removed in vacuo on a high vacuum pump, and the crude solid was dissolved in dichloromethane. The resulting precipitate was filtered off to give a beige powder (39.6 g, 92%) which was dried in vacuo. m.p. 185-186° C.; δ_{H} (MeOH-d₆) 1.47 (9H, s, O′Bu), 3.86 (2H, s, CH₂), 7.33 (1H, t J 7.9 Hz, ArH), 7.50 (1H, t J 6.9 Hz, ArH), 7.65 (1H, d J 8.5 Hz, ArH), 8.23 (1H, d J 8.2 Hz, ArH); δ_{C} (DMSO-d₆) 161.3, 157.6, 155.8, 140.8, 134.9, 126.6, 122.6, 122.1, 121.1, 110.9, 78.4, 40.9, 28.1; LC-MS-EI (MH⁺, 100); Found (FAB) 356.13284 C₁₅H₁₉N₅O₄Na (M+Na) requires 356.13347.

### PRODUCTION EXAMPLE 3

### Synthesis of t-butyl (5-(1H-indazol-3-yl)-1,2,41-oxadiazol-3 yl methyl carbamate

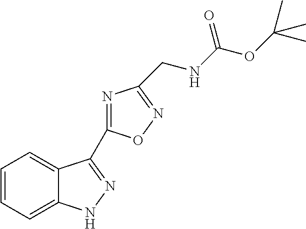

Into a 20 mL microwave vial was added the boc-protected compound name —O— acylated amidoxime from Production Example 2 (2 g, 6 mmol, 1 equiv.) in DMF (12 mL) and sodium acetate (0.54 g, 6.6 mmol, 1.1 equiv.). The microwave reaction was heated to 140° C. for 20 mins with stirring. The solvent was then removed in vacuo on a high vacuum pump and the remaining crude yellow solid was dissolved in dichloromethane (100 mL) and washed with water (100 mL), NaHCO₃ (aq) (100 mL), 1N HCl (aq) (100 mL) and brine (100 mL). The organic phase was then dried over magnesium sulfate and the solvent removed in vacuo to give a white powder (1.48 g, 78%); m.p. 169-170° C.; δ_{H} (MeOH-d₄) 1.47 (9H, s, O′Bu), 4.55 (2H, s, CH₂), 7.36 (1H, t J 6.9 Hz, ArH), 7.53 (1H, t J 5.9 Hz, ArH), 7.68 (1H, d J 8.5 Hz, ArH), 8.26 (1H, d J 7.3 Hz, ArH); δ_{C} (MeOH-d₆) 172.6, 158.3, 142.6, 131.8, 128.7, 124.4, 122.9, 121.9, 111.9, 80.8, 37.4, 28.7; LC-MS-EI 316.2 (MH⁺, 100); Found (FAB) 316.14004 C₁₅H₁₂N₅O₃ (MH⁺) requires 316.14096.

### EXAMPLE 1

### Synthesis of tert-butyl (5-(1-(2-(1H-imidazol-1-yl)ethyl)-1H-indazol-3-yl)-1,2,4-oxadiazol-3-yl)methylcarbamate

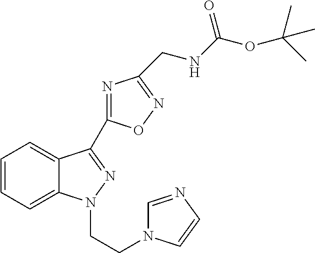

To a solution of t-butyl (5-(1H-indazol-3-yl)-1,2,4-oxadiazol-3 yl methyl carbamate (0.50 g, 1.59 mmol) in DMF (5 mL) was added cesium carbonate (1.55 g, 4.76 mmol, 3 equiv.) and the resulting mixture allowed to stir at room temperature under nitrogen for half an hour. N-(2-chloroethyl)imidazole hydrochloride (0.27 g, 1.59 mmol, 1 equiv.) was added and the solution allowed to stir overnight. The DMF was evaporated in vacuo and the crude oil dissolved in ethyl acetate and extracted with water and brine. The organic phase was dried over magnesium sulfate and the solvent was removed in vacuo. The crude yellow oil was then placed a top a flash column and eluted with 10% methanol in dichloromethane to give a clear oil (0.42 g, 65%); δ_{H} (300 MHz, CDCl₃) 1.48 (9H, s, (CH₃)₃), 4.59-4.63 (4H, m, 2×CH₂), 4.79 (2H, t J 5.8 Hz, NCH₂), 6.66 (1H, s, HC═CH), 6.92 (1H, s, HC═CH), 7.07-7.48 (4H, m, ArH+N═CH), 8.26 (1H, d J 8.2 Hz, ArH); δ_{C} (DMSO, 75.5 MHz); 170.0, 169.2, 155.6, 140.8, 137.3, 129.2, 128.4, 127.4, 123.6, 121.5, 120.3, 119.3, 110.5, 78.3, 49.8, 45.5, 36.0, 28.1; LC-MS-EI 410.4 (MN⁺, 100).

### EXAMPLE 2

### Synthesis of tert-butyl (5-(2-(2-(1H-imidazol-1-yl)ethyl)-2H-indazol-3-yl)-1,2,4-oxadiazol-3-yl)methylcarbamate

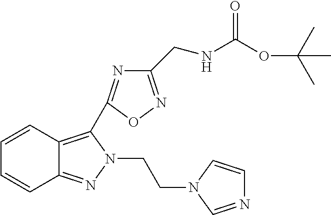

To a solution of t-butyl (5-(1H-indazol-3-yl)-1,2,4-oxadiazol-3 yl methyl carbamate (1.14 g, 3.6 mmol) in anhydrous toluene was added in order: 1-(2-hydroxyethyl) imidazole (0.43 g, 3.98 mmol, 1.1 equiv.), n-butyl phosphine (1.18 g, 1.44 mL, 5.79 mmol, 1.6 equiv.) and TMAD (1.0 g, 5.79 mmol), 1.6 equiv.) The clear orange solution was allowed to stir under nitrogen overnight at room temperature. The toluene was then evaporated off in vacuo and the crude orange oil dissolved in ethyl acetate and washed with water, 1 N HCl, 1N NaOH and brine. The organic phase was dried over magnesium sulfate and the solvent vacced off. The crude compound was purified by flash chromatography (10% methanol in dichloromethane) and then washed/stirred with diethyl ether to give a white solid (0.70 g, 47%); mp 100-101° C.; δ_{H} (300 MHz, MeOH-d₄); 1.47 (9H, s, (CH₃)₃)), 4.46 (2H, s, CH₂), 4.63 (2H, t J 5.8 Hz, CH₂), 5.30 (2H, t J 5.8 Hz, CH2), 6.67 (1H, s, ArH), 6.74 (1H, s, ArH), 7.05-7.39 (3H, m, ArH), 7.69 (1H, d J 8.6 Hz, ArH), 7.95 (1H, d J 8.3 Hz, ArH); δ_{C} (75 MHz, MeOH-d₄) 170.0, 169.0, 158.2, 149.5, 138.7, 129.1, 128.5, 126.9, 124.0, 121.3, 121.1, 121.0, 119.2, 80.8, 54.4, 47.8, 37.2, 23.2; LC-MS-EI 410.4 (MH⁺, 100);

### EXAMPLE 3

### Synthesis of (5-(1-(2-(1H-imidazol-1-yl)ethyl)-1H-indazol-3-yl)-1,2,4-oxadiazol-3-yl)methanamine

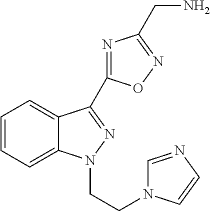

To tert-butyl (5-(1-(2-(1H-imidazol-1-yl)ethyl)-1H-indazol-3-yl)-1,2,4-oxadiazol-3-yl)methylcarbamate from Example 1 (0.42 g) was added trifluoroacetic acid (1.9 mL), tri-iso-propyl silane (0.05 mL) and water (0.05 mL) and the solution allowed to stir overnight at room temperature. The trifluoroacetic acid was then vacced off to give a clear oil (0.32 g, >99%); LC-MS-EI 310.4 (MH⁺, 46);

### EXAMPLE 4

### Synthesis of (5-(2-(2-(1H-imidazol-t-yl)ethyl)-2H-indazol-3-yl)-1,2,4-oxadiazol-3-yl)methanamine

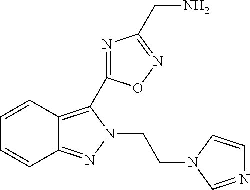

To (5-(2-(2-(1H-imidazol-1-yl)ethyl)-2H-indazol-3-yl)-1,2,4-oxadiazol-3-yl)methanamine from Example 2 (0.48 g) was added trifluoroacetic acid (3.8 mL), tri-iso-propyl silane (0.1 mL) and water (0.1 mL) and the solution allowed to stir overnight at room temperature. The trifluoroacetic acid was then vacced off and washed/stirred with diethyl ether to give a white powder (0.36 g, >99%); δ_{C} (75 MHz, MeOH-d₄) 170.1, 166.3, 149.6, 128.9, 127.6, 124.6, 121.2, 120.5, 119.4, 53.9, 35.9; mp 105-106° C.; LC-MS-EI 310.5 (MH⁺, 40); Found (CI) 310.14088, C₁₅H₁₅N₇O (MH⁺) requires 310.14108.

### EXAMPLE 5

### Synthesis of N-((5-(2-(2-(1H-imidazol-1-yl)ethyl)-2H-indazol-3-yl)-1,2,4-oxadiazol-3-yl)methyl)benzamide

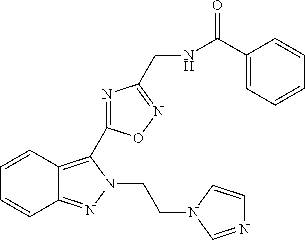

To a solution of benzoic acid (0.10 g, 0.82 mmol) in DMF (5 mL) was added HATU (0.34 g, 0.9 mmol, 1.1 equiv.) and di-iso-propyl ethyl amine (0.23 g, 0.31 mL, 1.8 mmol, 2.2 equiv.) and the solution allowed to stir for half an hour at room temperature under nitrogen. The amine (5-(2-(2-(1H-imidazol-1-yl)ethyl)-2H-indazol-3-yl)-1,2,4-oxadiazol-3-yl)methanamine from Example 5 (0.9 mmol, 1.1 equiv.) was then added and the brown solution allowed to stir overnight. The DMF was removed in vacuo and the resulting crude oil dissolved in ethyl acetate and washed with water, 1 N HCl, 1N NaHCO₃ and brine. The organic phase was then dried over magnesium sulfate and the solvent removed in vacuo. Unless otherwise stated, the crude oil was puffed using flash chromatography, eluting with 10% methanol in dichloromethane to obtain off-white powder (90 mg, 26%); mp 167-168° C.; δ_{H} (300 MHz, MeOH-d₄) 4.60 (1H, t J 6.0 Hz, NCH₂), 4.73 (1H, d J 5.8 Hz, CH₂NH), 5.30 (1H, t J 6.0 Hz, NCH₂), 6.67 (1H, s, NCH), 6.93 (1H, s, NCH), 7.33 (1H, s, NCHN), 7.39-7.56 (4H, m, ArH), 7.83 (1H, d J 8.3 Hz, ArH), 7.93 (2H, d J 8.4 Hz, ArH), 8.06 (1H, d J 7.9 Hz, ArH), 9.28 (1H, t J 5.7 Hz); δ_{C} (75 MHz, CDCl₃) 168.1, 167.6, 148.6, 137.6, 133.6, 131.9, 129.2, 128.6, 127.7, 127.4, 126.2, 123.0, 120.6, 120.1, 119.4, 118.3, 53.3, 47.1, 35.7; LC-MS-EI 414.2 (MH⁺, 100); Found (EI) 436.1520, C₂₂H₁₉N₇O₂Na (M+Na) requires 436.1498.

### EXAMPLE 6

### N-((5-(2-(2-(1H-imidazol-1-yl)ethyl)-2H-indazol-3-yl)-1,2,4-oxadiazol-3-yl)methyl)-2,3-dimethoxybenzamide

The above compound was prepared by analogous means to that used in Example 5.

### EXAMPLE 7

### N-((5-(2-(2-(1H-imidazol-1-yl)ethyl)-2H-indazol-3-yl)-1,2,4-oxadiazol-3-yl)methyl)-2,3-dimethoxybenzamide

The above compound was prepared by analogous means to that used in Example 5.

LC-MS-EI 428.4 (MH⁺, 100)

Cream powder

### EXAMPLE 8

### N-((5-(2-(2-(1H-imidazol-1-yl)ethyl)-2H-indazol-3-yl)-1,2,4-oxadiazol-3-yl)methyl)-4-acetamidobenzamide

The above compound was prepared by analogous means to that used in Example 5.

### EXAMPLE 9

### N-((5-(2-(2-(1H-imidazol-1-yl)ethyl)-2H-indazol-3-yl)-1,2,4-oxadiazol-3-yl)methyl)-4-chlorobenzamide

The above compound was prepared by analogous means to that used in Example 5.

### EXAMPLE 10

### N-((5-(2-(2-(1H-imidazol-1-yl)ethyl)-2H-indazol-3-yl)-1,2,4-oxadiazol-3-yl)methyl)-2,3-difluorobenzamide

The above compound was prepared by analogous means to that used in Example 5. Yellowy powder; Found (EI) 450.1474, C₂₂H₁₈N₇O₂F₂ (MH⁺) requires 450.1490.

### EXAMPLE 11

### N-((5-(2-(2-(1H-imidazol-1-yl)ethyl)-2H-indazol-3-yl)-1,2,4-oxadiazol-3-yl)methyl)-2-naphthamide

The above compound was prepared by analogous means to that used in Example 5.

White powder; LC-MS-EI 464.5 (MH⁺100); Found (EI) 464.1855, C₂₆H₂₂N₇O₂ (MH⁺) requires 464.1835.

### EXAMPLE 12

### N-((5-(2-(2-(1H-imidazol-1-yl)ethyl)-2H-indazol-3-yl)-1,2,4-oxadiazol-3-yl)methyl)-3-methyl-4-nitrobenzamide

The above compound was prepared by analogous means to that used in Example 5.

Feathery yellow powder; LC-MS-EI 473.4 (MH⁺, 100); Found (EI) 473.1666, C₂₃ H₂₁N₈O₄ (MH⁺) requires 473.1686.

### EXAMPLE 13

### N-((5-(2-(2-(1H-imidazol-1-yl)ethyl)-2H-indazol-3-yl)-1,2,4-oxadiazol-3-yl)methyl)benzo[d][1,3]dioxole-5-carboxamide

The above compound was prepared by analogous means to that used in Example 5.

White powder; LC-MS-EI 458.5 (MW, 100); Found (EI) 458.1589, C₂₃H₂₀N₇O₄ (MH⁺) requires 458.1577.

### EXAMPLE 14

### N-((5-(2-(2-(1H-imidazol-1-yl)ethyl)-2H-indazol-3-yl)-1,2,4-oxadiazol-3-yl)methyl)-3-(trifluoromethoxy)benzamide

The above compound was prepared by analogous means to that used in Example 5.

### EXAMPLE 15

N-((5-(1-(2-(1H-imidazol-1-yl)ethyl)-1H-indazol-3-yl)-1,2,4-oxadiazol-3-yl)methyl)benzamide

To a solution of benzoic acid in DMF (5 mL) was added HATU (1.1 equiv.) and di-iso-propyl ethyl amine (2.2 equiv.) and the solution allowed to stir for half an hour at room temperature under nitrogen. The amine (5-(1-(2-(1H-imidazol-1-yl)ethyl)-1H-indazol-3-yl)-1,2,4-oxadiazol-3-yl)methanamine from Example 3 (1.1 equiv.) was then added and the brown solution allowed to stir overnight. The DMF was vacced off in vacuo and the resulting crude oil dissolved in ethyl acetate and washed with water, 1 N HCl, 1N NaHCO3 and brine. The organic phase was then dried over magnesium sulfate and the solvent removed in vacuo. The crude oil was purified using flash chromatography and eluting with 10% methanol in dichloromethane to obtain orangey crystals; δ_{C} (75 MHz, CDCl₃) 170.1, 168.9, 166.5, 140.8, 137.2, 133.7, 131.5, 129.3, 128.4, 127.7, 127.4, 127.3, 123.7, 121.5, 120.3, 119.6, 110.5, 49.7, 45.7, 35.3; Found (EI) 414.1668, C₂₂H₂₀N₇O₂ (MH⁺) requires 414.1678.

### EXAMPLE 16

### N-((5-(1-(2-(1H-imidazol-1-yl)ethyl)-1H-indazol-3-yl)-1,2,4-oxadiazol-3-yl)methyl)-2,3,4-trimethoxybenzamide

The above compound was prepared by analogous means to that used in Example 15.

Yellow powder; Found (EI) 526.1840, C₂₅H₂₅N₇O₃ Na (M+Na) requires 526.1815.

### EXAMPLE 17

### N-((5-(1-(2-(1H-imidazol-1-yl)ethyl)-1H-indazol-3-yl)-1,2,4-oxadiazol-3-yl)methyl)-1H-indole-4-carboxamide

The above compound was prepared by analogous means to that used in Example 15.

### EXAMPLE 18

### N-((5-(1-(2-(1H-imidazol-1-yl)ethyl)-1H-indazol-3-yl)-1,2,4-oxadiazol-3-yl)methyl)-2,3-dimethoxybenzenesulfonamide

To a solution of N-((5-(1-(2-(1H-imidazol-1-yl)ethyl)-1H-indazol-3-yl)-1,2,4-oxadiazol-3-yl)methyl)benzamide in water (10 mL) was added 2,3-dimethyoxy benzene sulfonyl chloride. The solution was allowed to stir and saturated sodium carbonate (aq) was added gradually to adjust the pH to 8. The solution was then allowed to stir overnight at room temperature. The white precipitate was filtered off; washed with diethyl ether and dried in a genevac to obtain a white powder; 197-198° C.; LC-MS-EI 510.5 (MH⁺, 100); Found (EI) 532.1398, C₂₃H₂₃N₇O₅NaS (M+Na) requires 532.1379.

### EXAMPLE 19

### N-((5-(1-(2-(1H-imidazol-1yl)ethyl)-1H-indazol-3-yl)-1,2,4-oxadiazol-3-yl)methyl)-4-(trifluoromethoxy)benzenesulfonamide

The above compound was prepared by analogous means to that used in Example 18.

White powder; δ_{C} (75 MHz, CDCl₃) 170.0, 167.3, 150.7, 140.7, 139.4, 137.2, 131.0, 129.1, 129.0, 127.4, 127.1, 123.6, 121.5, 121.3, 100-101° C.; LC-MS-EI 534.5 (MH⁺, 100); Found (EI) 556.1008, C₂₂H₁₈N₇O₄NaSF₃ (M+Na) requires 556.0991.

### EXAMPLE 20

### N-(4-(N-((5-(2-(2-(1H-imidazol-1-yl)ethyl)-2H-indazol-3-yl)-1,2,4-oxadiazol-3-yl)methyl)sulfamoyl)phenyl)acetamide

To a solution of (5-(2-(2-(1H-imidazol-1-yl)ethyl)-2H-indazol-3-yl)-1,2,4-oxadiazol-3-yl)methanamine from Example 4 (0.22 g, 0.71 mmol, 1 equiv.) in water (10 mL) was added 4-(N-acetyl)-amino benzene sulfonyl chloride (0.78 mmol, 1.1 equiv.). The solution was allowed to stir and saturated sodium carbonate (aq) was added gradually to adjust the acidic pH to 8. The solution was then allowed to stir overnight at room temperature. The white precipitate was filtered off, washed with diethyl ether and dried in a genevac to give a white powder; δ_{H} (300 MHz, DMSO-d₆) 1.88 (3H, s, CH₃), 4.30 (2H, d J 5.9 Hz, CH₂NH), 4.57 (3H, t J 6.0 Hz, NCH₂), mp 160-161° C.;

### PRODUCTION EXAMPLE 4

### Synthesis of 5-fluoro-1H-indazole-3-carboxylic Acid

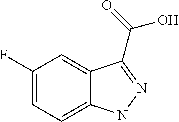

To a solution of NaOH (1.24 g, 0.031 mol, 1.03 equiv.) in water (19 mL) heated at 50° C. was added 5-Fluoroisatin (5 g, 0.03 mol, 1 equiv.). After 5 minutes, the dark-red solution was cooled to 0° C. and a cooled solution of sodium nitrite (2.07 g, 0.03 mol, 1 equiv.) in water (10 mL) was added slowly, followed by a cooled solution of (95%) (3.12 mL, 0.06 mol, 1.95 equiv.) in water (48 mL). The rate of addition was rapid such that the temperature never rose above 4° C. To reduce the foaming which occurred throughout the period of stirring, a few millilitres of ether were added when necessary. After the end of addition, the cooled solution was stirred for one hour, maintaining the temperature below 4° C. at all times. Then a cooled solution of SnCl (16.24 g, 0.072 mol, 2.4 equiv.) in HCl (35%) (24 mL) was added and the mixture was stirred for 16 h. The brown solution was filtrated and the resultion brown solid was washed with water to give a light brown solid (2.73 g, 51%); δ_{H} (MeOH-d₆) 7.25 (1H, td, J 9.10 Hz and J 2.14 Hz, ArH), 7.60 (1H, dd J 9.10 Hz and 4.28 Hz, ArH), 7.74 (1H, dd J 9.10 Hz and 2.10 Hz, ArH); LC-MS-EI 183.1 (MH⁺, 100)

### PRODUCTION EXAMPLE 5

### Synthesis of (Z)-tert-butyl 2-amino-2-(5-fluoro-1H-indazole-3-carbonyloxyimino)ethylcarbamate

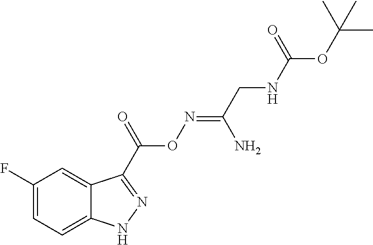

To a round bottomed flask was added 5-Fluoro-1H-indazole-3-carboxylic acid from Production Example 4 (2.73 g, 0.015 mol, 1 equiv.) in DMF (40 mL) under nitrogen. Carbodiimidazole (CDI) (2.98 g, 0.018 mol, 1.1 equiv.) was added to the resulting yellow solution and this was stirred at room temperature for 30 minutes. N-(tert-butoxycarbonyl)-2-aminoacetamidoxime (3.46 g, 0.018 mol, 1.1 equiv.) in DMF (20 mL) was then added and the solution was stirred overnight at room temperature. The solvent was removed in vacuo on a high vacuum pump, and the crude solid was dissolved in dichloromethane. The resulting precipitate was filtered off and dried in in vacuo to give a light brown powder (4.04 g, 75%); LC-MS-EI 352.4 (MH⁺, 100).

### PRODUCTION EXAMPLE 6

### Synthesis of tert-butyl (5-(5-fluoro R-1H-indazol-3-yl)-1,2,4-oxadiazol-3-yl)methylcarbamate

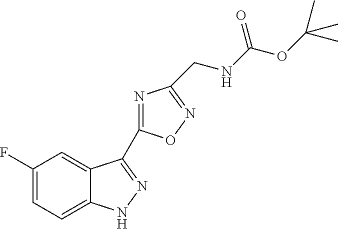

Into a 20 mL microwave vial was added (Z)-tert-butyl 2-amino-2-(5-fluoro-1H-indazole-3-carbonyloxyimino)ethylcarbamate from Production Example 5 (2 g, 5.70 mmol, 1 equiv.) in DMF (12 mL) and sodium acetate (0.51 g, 6.3 mmol, 1.1 equiv.). The microwave reaction was heated to 140° C. for 20 mins with stirring. The solvent was then removed in vacuo on a high vacuum pump and the remaining crude yellow solid was dissolved in dichloromethane (100 mL) and washed with water (100 mL), NaHCO₃ (aq) (100 mL), 1N HCl (aq) (100 mL) and brine (100 mL). The organic phase was then dried over magnesium sulfate and the solvent removed in vacuo to give a white powder (1.5 g, 79%); LC-MS-EI 316.2 (MH⁺, 100).

### EXAMPLE 21

### Synthesis of tert-butyl (5-(2-(2-(1H-imidazol-1-yl)ethyl)-5-fluoro-2H-indazol-3-yl)-1,2,4-oxadiazol-3-yl)methylcarbamate

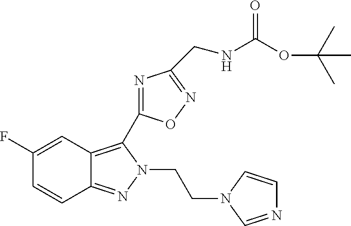

To a solution of tert-butyl (5-(5-fluoro-1H-indazol-3-yl)-1,2,4-oxadiazol-3-yl)methylcarbamate (1.5 g, 4.50 mmol, 1 equiv.) in anhydrous toluene was added in order: 1-(2-hydroxyethyl) imidazole (0.55 g, 4.95 mmol, 1.1 equiv.), n-butyl phosphine (1.78 mL, 7.2 mmol, 1.6 equiv.) and TMAD (1.24 g, 7.2 mmol, 1.6 equiv.). The clear orange solution was allowed to stir under nitrogen overnight at room temperature. The toluene was then evaporated off in vacuo and the crude orange oil dissolved in ethyl acetate and washed with water, 1 N HCl, 1N NaOH and brine. The organic phase was dried over magnesium sulfate and the solvent vacced off.

### EXAMPLE 22

### Synthesis of (5-(2-(2-(1H-imidazol-1-yl)ethyl)-5-fluoro-2H-indazol-3-yl)-1,2,4-oxadiazol-3-yl)methanamine

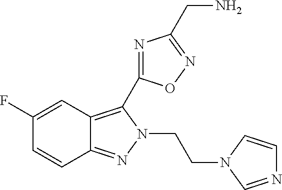

To the crude tert-butyl (5-(2-(2-(1H-imidazol-1-yl)ethyl)-5-fluoro-2H-indazol-3-yl)-1,2,4-oxadiazol-3-yl)methylcarbamate from Example 21 (0.64 g) was added trifluoroacetic acid (3.8 mL), tri-iso-propyl silane (0.1 mL) and water (0.1 mL) and the solution allowed to stir overnight at room temperature. The trifluoroacetic acid was then vacced off and washed/stirred with diethyl ether to give 0.3 g of a yellow oil, which was purified using flash chromatography, eluting with 10% methanol in dichloromethane, giving an orange oil (0.227 g, 48%); δ_{H} (300 MHz, MEOH-d₄) 4.46 (2H, m, CH₂), 4.96 (2H, t J 5.50 Hz, NCH₂CH₂N), 5.50 (2H, t J 5.19 Hz, NCH₂CH₂N), 7.28 (2H, m, HC═CH), 7.39 (1H, s, N═CH), 7.74 (2H, m, ArH), 8.63 (1H, m, ArH); LC-MS-EI 328.5 (MH⁺, 100); Found (ESI) 328.1322 C₁₅H₁₅FN₇O (M+H) requires 328.1319.

### EXAMPLE 23

### N-((5-(2-(2-(1H-imidazol-1-yl)ethyl)-5-fluoro-2H-indazol-3-yl)-1,2,4-oxadiazol-3-yl)methyl)-2,3-dimethoxybenzamide

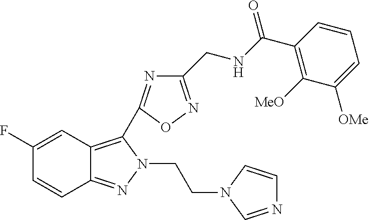

To a solution of R-substituted benzoic acid (0.13 g, 0.69 mmol, 1 equiv.) in DMF (5 mL) was added HATU (0.29 g, 0.76 mmol, 1.1 equiv.) and di-iso-propyl ethyl amine (0.27 mL, 1.53 mmol, 2.2 equiv.) and the solution allowed to stir for half an hour at room temperature under nitrogen. The amine (5-(2-(2-(1H-imidazol-1-yl)ethyl)-5-fluoro-2H-indazol-3-yl)-1,2,4-oxadiazol-3-yl)methanamine from Example 22 (0.227 g, 0.69 mmol, 1 equiv.) was then added and the brown solution allowed to stir overnight. The DMF was removed in vacuo and the resulting crude oil dissolved in ethyl acetate and washed with water, 1 N HCl, 1N NaHCO₃ and brine. The organic phase was then dried over magnesium sulfate and the solvent removed in vacuo. The crude oil was purifed using flash chromatography, eluting with 10% methanol in dichloromethane giving a yellow oil (0.01 g, 3%); δ_{H} (300 MHz, MEOH-d₄) 3.85 (3H, m, OCH₃), 3.95 (3H, s, OCH₃), 4.81 (2H, m, CH₂), 4.89 (2H, m, NCH₂CH₂N), 5.52 (2H, m, NCH₂CH₂N), 6.88 (1H, s, HC═CH), 6.98 (1H, s, HC═CH), 7.21 (3H, m, ArH), 7.42 (1H, s, N═CH), 7.63 (2H, m, ArH), 7.77 (1H, m, ArH); LC-MS-EI 492.8 (MH⁺, 100); Found (ESI) 492.1796 C₂₄H₂₃FN₇O₄ (M+H) requires 492.1784.

### EXAMPLE 24

### N-((5-(2-(2-(1H-imidazol-1-yl)ethyl)-2H-indazol-3-yl)-1,2,4-oxadiazol-3-yl)methyl)-3-methylbenzamide

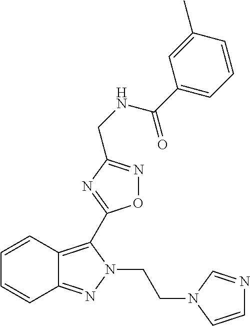

The above compound was prepared by analogous means to those used in the foregoing examples

White solid (0.05g, 20%); δ_{H} (300 MHz, CDCl₃) 2.33 (3H, s, CH₃), 4.50 (2H, t J 5.88 Hz, NCH₂CH₂N), 4.82 (2H, d J 7.22 Hz, NCH₂), 5.31 (2H, t J 5.88 Hz, NCH₂CH₂N), 6.41 (1H, s, HC═CH), 6.72 (1H, s, HC═CH), 7.37 (4H, m, ArH+HC═N), 7.84 (3H, m, ArH), 8.03 (2H, m, ArH); δ_{C} (75.5 MHz, CDC1₃) 167.82, 167.82, 167.79, 148.52, 138.49, 133.56, 132.62, 128.92, 128.49, 128.11, 127.67, 126.15, 124.34, 122.94, 120.53, 120.06, 119.37, 118.32, 53.24, 46.96, 35.70, 29.70, 21.33; LC-MS-EI 428.4 (MH⁺, 100); Found (ESI) 450.16544, C₂₃H₂₁N₇O₂ (M+Na) requires 450.16635.

### EXAMPLE 25

### N-((5-(2-(2-(1H-imidazol-1-yl)ethyl)-2H-indazol-3-yl)-1,2,4-oxadiazol-3-yl)methyl)benzenesulfonamide

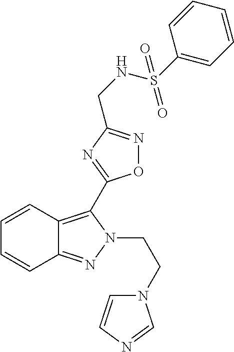

The above compound was prepared by analogous means to those used in the foregoing Examples.

White powder (0.122 g, 28%); δ_{H} (300 MHz, DMSO-d₆) 4.33 (2H, s, NCH₂), 4.58 (2H, t J 5.91 Hz, NCH₂CH₂N), 5.23 (2H, t J 5.88 Hz, NCH₂CH₂N), 6.72 (1H, s, HC═CH), 6.94 (1H, s, HC═CH), 7.32 (1H, s, HC═N), 7.47 (5H, m, ArH), 7.84 (3H, m, ArH), 8.12 (1H, d J 7.89 Hz, ArH), 8.65 (1H, br s, NH); δ_{C} (75.5 MHz, DMSO-d₆) 167.072, 167.00, 147.29, 140.55, 137.36, 132.26, 128.92, 128.92, 128.31, 127.11, 126.47, 126.47, 125.73, 122.24, 119.97, 119.42, 118.98, 118.22, 53.25, 45.57, 37.81; LC-MS-EI 450.5 (MH⁺, 100); Found (EST) 472.11633, C₂₁H₁₉N₇O₃S (M+Na) requires 472.11678.

### EXAMPLE 26

### N-((5-(1-(2-(1H-imidazol-1-yl)ethyl)-1H-indazol-3-yl)-1,2,4-oxadiazol-3-yl)methyl)-2-methylbenzenesulfonamide

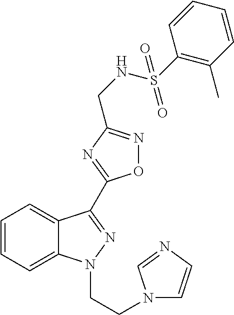

The above compound was prepared by analogous means to those used in the foregoing examples.

White powder (0.145 g, 39%); δ_{H} (300 MHz, MeOH-d₄) 2.58 (3H, s, CH₃), 4.32 (2H, s, NCH₂), 4.76 (2H, t J 5.19 Hz, NCH₂CH₂N), 4.97 (2H, t J 4.95 Hz, NCH₂CH₂N), 7.07 (1H, s, HC═CH), 7.09 (1H, s, HC═CH), 7.39 (3H, m, HC═N+ArH), 7.48 (2H, m, ArH), 7.89 (3H, m, ArH), 8.12 (2H, d J 4.31 Hz, ArH), 8.65 (1H, br s, NH); δ_{C} (75.5 MHz, DMSO-d₆) 169.78, 167.36, 146.20, 138.35, 135.45, 132.33, 132.10, 130.57, 129.16, 127.52, 126.42, 125.87, 124.97, 124.66, 123.61, 121.50, 120.48, 110.47, 49.32, 46.50, 37.40, 19.83; LC-MS-EI 464.4 (MH⁺, 100); Found (ESI) 464.15096, C₂₂H₂₁N₇O₃S (M+H) requires 464.15048.

### EXAMPLE 27

### N-((5-(1-(2-(1H-imidazol-1-yl)ethyl)-1H-indazol-3-yl)-1,2,4-oxadiazol-3-yl)methyl)-3-(trifluoromethyl)benzenesulfonamide

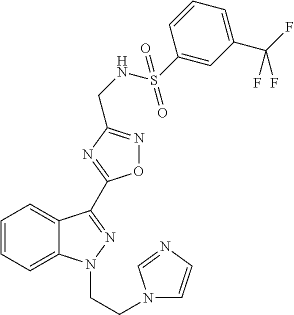

The above compound was prepared by analogous means to those used in the foregoing examples.

White powder (0.143 g, 34%); δ_{H} (300 MHz, MeOH-d₄) 4.47 (2H, s, NCH₂), 4.79 (2H, t J 5.11 Hz, NCH₂CH₂N), 5.00 (2H, t J 4.88 Hz, NCH₂CH₂N), 7.15 (1H, s, HC═CH), 7.25 (1H, s, HC═CH), 7.37 (3H, m, HC═N+ArH), 7.64 (3H, m, ArH), 8.10 (4H, m, ArH); δ_{C} (75.5 MHz, MeOH-d₄) 172.04, 168.65, 143.45, 142.48, 137.73, 131.80, 131.45, 131.45, 131.32, 130.69, 130.51, 130.20, 129.15, 127.84, 124.96, 123.87, 123.42, 122.29, 110.74, 50.53, 48.19, 39.18; LC-MS-EI 518.4 (MH⁺, 100); Found (ESI) 518.12347, C₂₂H₁₈F₃N₇O₃S (M+H) requires 518.12222.

### EXAMPLE 28

### N-((5-(1-(2-(1H-imidazol-1-yl)ethyl)-1H-indazol-3-yl)-1,2,4-oxadiazol-3-yl)methyl)-4-tert-butylbenzenesulfonamide

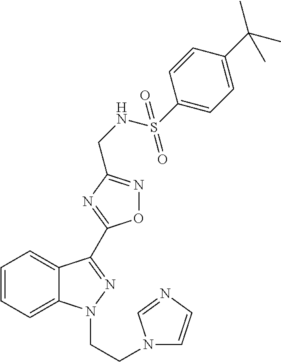

The above compound was prepared by analogous means to those used in the foregoing examples

White powder (0.099 g, 24%); δ_{H} (300 MHz, MeOH-d₄) 1.31 (9H, s, CH₃), 4.39 (2H, s, NCH₂), 4.67 (2H, t J 5.66 Hz, NCH₂CH₂N), 4.88 (2H, m, NCH₂CH₂N), 6.84 (1H, s, HC═CH), 6.90 (1H, s, HC═CH), 7.31 (2H, m, HC═N+ArH), 7.44 (3H, m, ArH), 7.74 (3H, m, ArH), 8.08 (1H, d J 8.10 Hz, ArH); δ_{C} (75.5 MHz, DMSO-d₆) 169.78, 167.22, 155.18, 150.96, 145.25, 140.78, 137.44, 129.07, 127.49, 126.38, 125.57, 125.26, 125.26, 124.28, 124.28, 123.60, 121.52, 110.49, 49.64, 47.50, 37.68, 34.26, 31.02, 31.02 30.351; LC-MS-EI 506.4 (MH⁺, 100); Found (ESI) 506.19612, C₂₅H₂₇N₇O₃S (M+H) requires 506.19743.

### EXAMPLE 29

### N-((5-(1-(2-(1H-imidazol-1-yl)ethyl)-1H-indazol-3-yl)-1,2,4-oxadiazol-3-yl)methyl)-4-tert-butylbenzamide

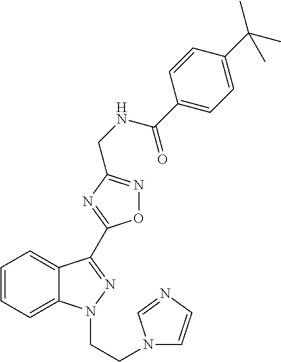

The above compound was prepared by analogous means to those used in the foregoing examples

White solid (0.049 g, 25%); δ_{H} (300 MHz, MeOH-d₄) 1.32 (9H, s, CH₃), 4.62 (2H, m, NCH₂), 4.81 (2H, m, NCH₂CH₂N), 4.87 (2H, m, NCH₂CH₂N), 6.76 (1H, s, HC═CH), 6.91 (1H, s, HC═CH), 7.34 (4H, m, HC═N+ArH), 7.51 (2H, d J 8.70 Hz, ArH 7.86 (2H, m, ArH), 8.12 (1H, d J 8.09 Hz, ArH); δ_{C} (75.5 MHz, MeOH-d₄) 172.26, 170.39, 170.07, 156.71, 142.66, 138.44, 132.19, 131.54, 128.92, 128.85, 128.85, 128.42, 126.58, 126.58, 124.82, 123.45, 122.13, 120.86, 110.71, 51.14, 47.56, 36.71, 35.70, 35.82, 31.57; LC-MS-EI 470.5 (MH⁺, 100); Found (ESI) 492.21281, C₂₆H₂₇N₇O₂ (M+Na) requires 492.21239

### EXAMPLE 30

### N-((5-(1-(2-(1H-imidazol-1-yl)ethyl)-1H-indazol-3-yl)-1,2,4-oxadiazol-3-yl)methyl)-2,3-dimethoxybenzamide

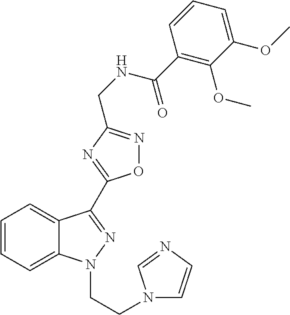

The above compound was prepared by analogous means to those used in the foregoing examples

White solid (0.132 g, 35%); δ_{H} (300 MHz, MeOH-d₄) 3.84 (6H, s, OCH₃), 4.67 (2H, m, NCH₂), 4.84 (2H, m, NCH₂CH₂N), 5.47 (2H, m, NCH₂CH₂N), 6.86 (1H, s, HC═CH), 7.00 (1H, s, HC═CH), 7.15 (6H, m, HC═N+ArH), 7.53 (1H, s, ArH), 8.05 (1H, d J 8.10 Hz, ArH); δ_{C} (75.5 MHz, DMSO-d₆) 170.04, 168.82, 167.80, 165.54, 152.95, 152.55, 147.41, 146.74, 140.81, 129.24, 127.40, 124.06, 123.78, 121.53, 121.03, 120.31, 119.33, 115.25, 110.49, 60.72, 55.79, 49.82, 45.49, 35.25; LC-MS-EI 474.4 (MH⁺, 100); Found (EI) 496.1722, C₂₄H₂₃N₇O₄ (M+Na) requires 496.1709.

### EXAMPLE 31

### N-((5-(1-(2-(1H-imidazol-1-yl)ethyl)-1H-indazol-3-yl)-1,2,4-oxadiazol-3-yl)methyl)-3-(trifluoromethoxy)benzamide

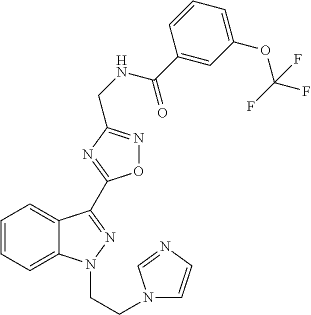

The above compound was prepared by analogous means to those used in the foregoing examples

White powder (0.036 g, 9%); δ_{H} (300 MHz, MeOH-d₄) 4.66 (2H, m, NCH₂), 4.83 (2H, m, NCH₂CH₂N), 5.02 (2H, m, NCH₂CH₂N), 6.75 (1H, s, HC═CH), 6.90 (1H, s, HC═CH), 7.41 (5H, m, HC═N+ArH), 7.60 (1H, t J 8.06 Hz, ArH), 7.83 (1H, s, ArH), 7.90 (1H, s, ArH), 7.92 (1H, d J 8.10 Hz, ArH), 8.20 (1H, d J 8.11 Hz, ArH); δ_{C} (75.5 MHz, DMSO-d₆) 170.14, 168.64, 164.90, 148.31, 140.82, 135.80, 130.68, 129.18, 127.41, 126.42, 126.42, 124.14, 124.14, 123.67, 123.67, 121.51, 120.29, 119.78, 118.29, 110.54, 49.83, 45.47, 35.29; LC-MS-EI 498.4 (MH⁺, 100); Found (ESI) 498.15124, C₂₃H₁₈F₃N₇O₃ (M+H) requires 498.15015.

### EXAMPLE 32

### tert-butyl (5-(1-methylthiazol-4-yl)methyl)-1H-indazol-3-yl)-1,2,4-oxadiazol-3-yl)methylcarbamate

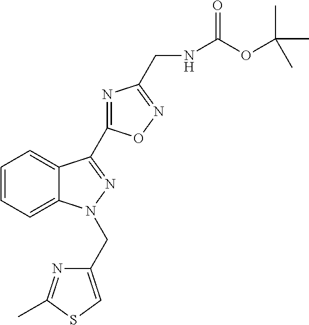

The above compound was prepared by analogous means to those used in the foregoing examples

Yellow solid (0.353 g, 52%); δ_{H} (300 MHz, DMSO-d₆) 1.45 (9H, s, O′Bu), 2.49 (3H, m, CH₃), 4.37 (2H, d J 5.95 Hz, NCH₂), 5.87 (2H, in, CH₂), 7.42 (1H, t J 7.65 Hz, ArH), 7.55 (1H, s, CH), 7.57 (1H, t J 7.52 Hz, ArH), 7.96 (1H, d J 8.53 Hz, ArH), 8.18 (1H, d J 8.14 Hz, ArH); δ_{C} (75.5 MHz, DMSO-d₆) 169.97, 169.16, 166.29, 155.59, 150.01, 140.42, 128.86, 127.47, 123.65, 121.93, 120.46, 117.59, 111.45, 78.31, 49.17, 36.031, 28.11, 18.64; LC-MS-EI 427.4 (MH⁺, 100); Found (ESI) 449.13846 C₂₀H₂₂N₆O₃S (M+Na) requires 449.13718.

### EXAMPLE 33

### (5-(1-((2-methylthiazol-4-yl)methyl)-1H-indazol-3-yl)-1,2,4-oxadiazol-3-yl)methanamine

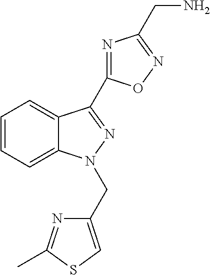

The above compound was prepared by analogous means to those used in the foregoing examples.

White solid (0.294 g, >99%); δ_{H} (300 MHz, DMSO-d₆) 2.55 (3H, s, CH₃), 4.45 (2H, s, NCH₂), 5.89 (2H, s, CH₂), 7.46 (1H, t J 8.03 Hz, ArH), 7.55 (1H, s, CH), 7.61 (1H, t J 8.03 Hz, ArH), 8.00 (1H, d J 8.57 Hz, ArH), 8.25 (1H, d J 8.03 Hz, ArH)) 8.76 (2H, br s, NH₂); δ_{C} (75.5 MHz, DMSO-d₆) 170.67, 166.35, 165.66, 149.88, 140.49, 128.39, 127.63, 123.91, 122.02, 120.44, 117.78, 111.61, 49.25, 34.21, 18.65; LC-MS-EI 327.3 (MH⁺, 100); Found (EI) 326.09376 C₁₅H₁₄N₆OS (M+H) requires 326.09443.

### EXAMPLE 34

### tert-butyl (5-(2-(2-(thiophen-2-yl)ethyl)-2H-indazol-3-yl)-1,2,4-oxadiazol-3-yl)methylcarbamate

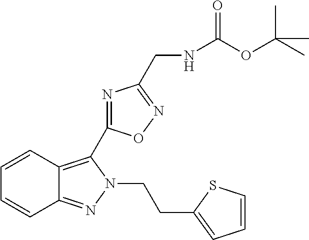

The above compound was prepared by analogous means to those used in the foregoing examples.

White solid (0.210 g, 31%); δ_{H} (300 MHz, DMSO-d₆) 1.39 (9H, s, O′Bu), 3.51 (2H, t J 7.19 Hz, CH₂CH₂N), 4.39 (2H, d J 5.95 Hz, NCH₂), 5.20 (2H, d J 7.04 Hz, NCH₂CH₂), 6.88 (2H, m, HC═CH), 7.43 (2H, m, ArH+HC═CH), 7.60 (1H, m, ArH), 7.85 (1H, d J 8.57 Hz, ArH), 8.08 (1H, d J 8.03 Hz, ArH); δ_{C} (75.5 MHz, DMSO-d₆) 168.84, 167.31, 155.58, 147.21, 139.02, 126.93, 126.93, 126.04, 125.50, 124.64, 122.35, 119.99, 118.86, 118.18, 78.34, 53.97, 36.01, 29.82, 28.10; LC-MS-EI 426.3 (MH⁺, 100); Found (FAB) 448.14225 C₂₁H₂₃N₅O₃S (M+Na) requires 448.14192.

### EXAMPLE 35

### N-((5-(1-((2-methylthiazol-4-yl)methyl)-1H-indazol-3-yl)-1,2,4-oxadiazol-3-yl)methyl)benzamide

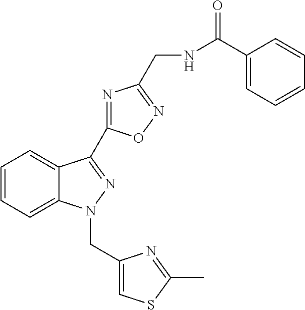

The above compound was prepared by analogous means to those used in the foregoing examples.

White solid (0.205 g, 75%); δ_{H} (300 MHz, DMSO-d₆) 2.55 (3H, s, CH), 4.72 (2H, d J 5.73 Hz, NCH₂), 5.86 (2H, s, CH₂), 7.47 (6H, m, CH+ArH), 7.93 (3H, m, ArH), 8.16 (1H, d J 8.19 Hz, ArH), 9.26 (1H, t J 5.70 Hz, NH); δ_{C} (75.5 MHz, DMSO-d₆) 170.15, 168.86, 166.46, 166.29, 150.01, 140.42, 133.67, 131.49, 128.84, 128.35, 127.47, 127.28, 123.72, 121.92, 120.43, 117.57, 111.46, 49.17, 35.23, 18.64; LC-MS-EI 431.3 (MH⁺, 100); Found (CI) 431.12984 C₂₂H₁₈N₆O₂S (M+H) requires 431.12901.

### EXAMPLE 36

### (5-(2-(2-(thiophen-2-yl)ethyl)-2H-indazol-3-yl)-1,2,4-oxadiazol-3-yl)methanamine

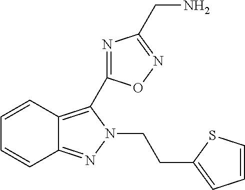

The above compound was prepared by analogous means to those used in the foregoing examples.

White powder (0.124 g, 75%); δ_{H} (300 MHz, DMSO-d₆) 3.55 (2H, t J 7.02 Hz, CH₂CH₂N), 4.47 (2H, s, NCH₂), 5.24 (2H, t J 7.06 Hz, NCH₂CH₂), 6.86 (2H, m, HC═CH), 7.29 (1H, m, HC═CH), 7.46 (2H, m, ArH), 7.89 (1H, d J 7.50 Hz, ArH), 8.23 (1H, d J 7.50 Hz, ArH), 8.77 (2H, br s, NH2); δ_{C} (75.5 MHz, DMSO-d₆) 168.83, 165.32, 147.27, 139.03, 127.07, 126.78, 126.07, 125.84, 124.69, 122.60, 119.97, 118.51, 118.33, 54.07, 34.26, 29.78; LC-MS-EI 326.3 (MH⁺, 100); Found (EI) 326.1076 C₁₆H₁₅N₅OS (M+H) requires 326.1053.

### EXAMPLE 37

### N-((5-(2-(2-(thiophen-2-yl)ethyl)-2H-indazol-3-yl)-1,2,4-oxadiazol-3-yl)methyl)benzamide

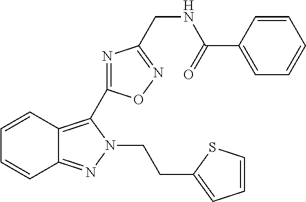

The above compound was prepared by analogous means to those used in the foregoing examples

White powder (0.048 g, 40%); δ_{H} (300 MHz, DMSO-d₆) 3.48 (2H, t J 7.34 Hz, CH₂CH₂N), 4.74 (2H, d J 5.73 Hz, NCH₂), 5.20 (2H, t J 7.17 Hz, NCH₂CH₂), 6.80 (2H, m, HC═CH), 7.20 (1H, m, HC═CH), 7.48 (5H, m, ArH), 7.91 (3H, m, ArH), 8.08 (1H, d 7.98 Hz, ArH), 9.27 (1H, t J 5.68 Hz, NH); δ_{C} (75.5 MHz, DMSO-d₆) 168.55, 166.50, 147.24, 138.98, 138.98, 133.68, 131.51, 128.36, 128.36, 127.29, 126.89, 126.89, 125.98, 125.58, 124.59, 122.36, 119.96, 118.83, 118.21, 114.75, 53.96, 35.14, 29.85; LC-MS-EI 430.3 (MH⁺, 100); Found (CI) 430.13256 C₂₃H₁₉N₅O₂S (M+H) requires 430.13376.

### EXAMPLE 38

### tert-butyl (5-(2-((pyridin-2-yl)methyl)-2H-indazol-3-yl)-1,2,4-oxadiazol-3-yl)methylcarbamate

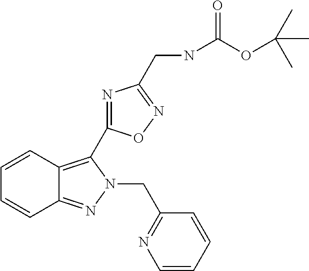

The above compound was prepared by analogous means to those used in the foregoing examples

White solid (0.375 g, 73%); δ_{H} (300 MHz, DMSO-d₆) 1.45 (9H-isomer N1+9H-isomer N2, s, O′Bu-isomer N1+N2), 4.33 (2H, d J 6.06 Hz, NCH₂-isomer N2), 4.37 (2H, d J 5.94 Hz, NCH₂-isomer N1), 5.96 (2H, s, CH₂-isomer N2), 6.34 (2H, s, CH₂-isomer N1), 7.29 (3H, m, ArH-isomer N2), 7.43 (2H, m, ArH-isomer N2), 7.56 (6H, m, ArH-isomer N1), 7.74 (2H, m, ArH-isomer N2), 7.76 (1H, d J 8.57 Hz, ArH-isomer N2), 8.16 (1H, d J 8.031 Hz, ArH-isomer N2), 8.20 (1H, d J 8.031 Hz, ArH-isomer N1), 8.40 (1H, d J 4.82 Hz, ArH-isomer N1), 8.48 (1H, d J 4.82 Hz, ArH-isomer N2); δ_{C} (75.5 MHz, DMSO-d₆); LC-MS-EI 407.3 (MH⁺, 100); Found (EI) 406.17039 C₂₁H₂₂N₆O₃ (M+H) requires 406.16983.

### EXAMPLE 39

### (5-(2-((pyridin-2-yl)methyl)-2H-indazol-3-yl)-1,2,4-oxadiazol-3-yl)methanamine (70% N2+30% N1)

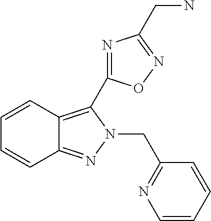

The above compound was prepared by analogous means to those used in the foregoing examples

White powder (0.200 g, >99%); δ_{H} (300 MHz, DMSO-d₆) 4.64 (2H-isomer N1+2H-isomer N2, m, NCH₂-isomer N1+N2), 5.99 (2H, s, CH₂-isomer N2), 6.38 (2H, s, CH₂-isomer N1), 7.32 (3H, m, ArH-isomer N2), 7.48 (2H, m, ArH-isomer N2), 7.59 (1H, t J 7.50 Hz, ArH-isomer N2), 7.85 (6H, m, ArH-isomer N1), 8.23 (1H, m, ArH-isomer N2), 8.27 (1H, d J 8.03 Hz, ArH-isomer N1), 8.39 (1H, d J 4.82 Hz, ArH-isomer N1), 8.48 (1H, d J 4.82 Hz, ArH-isomer N2), 8.75 (2H-isomer N1+2H-isomer N2, bs, NH₂-isomer N1+N2); δ_{C} (75.5 MHz, DMSO-d₆) 155.33, 149.32, 149.32, 141.02, 134.83, 137.35, 127.76, 126.78, 123.92, 123.14, 122.07, 120.45, 110.88, 54.54, 34.22; LC-MS-EI 307.4 (MH⁺, 100); Found (EI) 306.11785 C₁₆H₁₄N₆O (M+H) requires 306.11740.

### EXAMPLE 40

### N-((5-(2-((pyridin-2-yl)methyl)-2H-indazol-3-yl)-1,2,4-oxadiazol-3-yl)methyl)benzamide

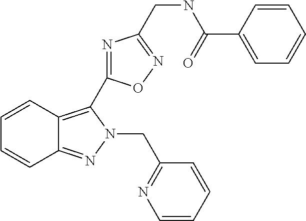

The above compound was prepared by analogous means to those used in the foregoing examples.

White powder (0.065 g, 24%); δ_{H} (300 MHz, DMSO-d₆) 4.67 (2H, d J 5.81 Hz, NCH₂-isomer N1), 4.72 (2H, d J 5.70 Hz, NCH₂-isomer N2), 5.96 (2H, s, CH₂-isomer N2), 6.32 (2H, s, CH₂-isomer N1), 7.26 (2H, in, ArH-isomer N2), 7.30 (4H, m, ArH-isomer N1), 7.49 (7H, m, ArH-isomer N2), 7.76 (1H, m, ArH-isomer N2), 7.84 (1H-isomer N1+1H-isomer N2, m, ArH-isomer N1+N2), 7.90 (2H-isomer N2+4H-isomer N1, t J 7.50 Hz, ArH-isomer N1+N2), 8.17 (2H-isomer N1+1H-isomer N2, m, ArH-isomer N1+N2), 8.35 (1H, d J 5.35 Hz, ArH-isomer N1), 8.47 (1H, d J 4.82 Hz, ArH-isomer N2), 9.22 (1H, t J 5.89 Hz, NH-isomer Ni), 9.27 (1H, t J 5.89 Hz, NH-isomer N2); δ_{C} (75.5 MHz, DMSO-d₆) 170.14, 168.87, 166.47, 155.52, 149.36, 149.36, 140.93, 137.20, 133,66, 131.49, 129.10, 128.35, 128.35, 127.59, 127.28, 127.28, 123.73, 123.02, 121.88, 120.45, 111.40, 54.51, 35.23; LC-MS-EI 411.3 (MH⁺, 100); Found (CI) 411.15692 C₂₃H₁₈N₆O₂ (M+H) requires 411.15695.

### EXAMPLE 41

### N-((5-(2-(2-(1H-imidazol-1-yl)ethyl)-2H-indazol-3-yl)-1,2,4-oxadiazol-3-yl)methyl)-2-methoxybenzamide

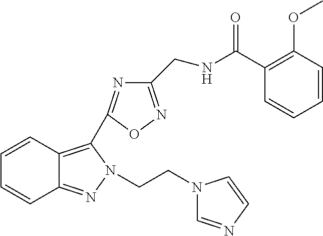

The above compound was prepared by analogous means to those used in the foregoing examples.

White solid (0.071 g, 20%); δ_{H} (300 MHz, DMSO-d₆) 3.93 (3H, s, OCH₃), 4.73 (4H, d J 5.35 Hz, NCH₂), 4.78 (2H, d J 5.89 Hz, NCH₂CH₂N), 5.36 (2H, t J 5.89 Hz, NCH₂CH₂N), 7.06 (2H, m, HC═CH), 7.19 (1H, d J 8.03 Hz, ArH), 7.26 (1H, s, N═CH), 7.46 (3H, m, ArH), 7.82 (2H, m, ArH), 8.04 (1H, s, ArH), 8.10 (1H, m, ArH), 8.93 (1H, t J 5.89 Hz, NH); δ_{C} (75.5 MHz, DMSO-d₆); LC-MS-EI 444.5 (MH⁺, 100); Found (CI) 444.17988, C₂₃H₂₁N₇O₃ (M+H) requires 444.17841.

### EXAMPLE 42

### N-((5(2-(2-(1H-imidazol-1-yl)ethyl)-2H-indazol-3-yl)-1,2,4-oxadiazol-3-yl)methyl)-3-methoxybenzamide

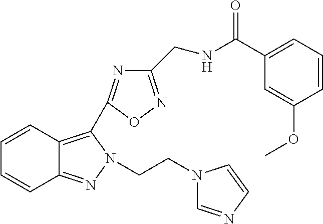

The above compound was prepared by analogous means to those used in the foregoing examples.

White solid (0.063 g, 18%); δ_{H} (300 MHz, DMSO-d₆) 3.80 (3H, s, OCH₃), 4.60 (2H, d J 5.89 Hz, NCH₂), 4.73 (2H, d J 5.89 Hz, NCH₂CH₂N), 5.30 (2H, t J 5.89 Hz, NCH₂CH₂N), 6.66 (1H, s, HC═CH), 6.93 (1H, s, HC═CH), 7.12(1H, m, ArH), 7.31 (1H, s, N═CH), 7.45 (5H, m, ArH), 7.84 (1H, d J 8.57 Hz, ArH), 8.07 (1H, d J 8.03 Hz, ArH), 9.28 (1H, t J 5.89 Hz, NH); δ_{C} (75.5 MHz, DMSO-d₆); LC-MS-EI 444.5 (MH⁺, 100); Found (CI) 444.17863, C₂₃H₂₁N₇O₃ (M+H) requires 444.17841.

### EXAMPLE 43

### N-((5-(2-(2-(1H-imidazol-1-yl)ethyl)-2H-indazol-3-yl)-1,2,4-oxadiazol-3-yl)methyl)-4-methoxybenzamide

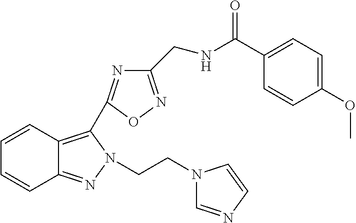

The above compound was prepared by analogous means to those used in the foregoing examples.

White solid (0.063 g, 18%); δ_{H} (300 MHz, DMSO-d₆) 3.81 (311, s, OCH₃), 4.60 (2H, t J 5.89 Hz, NCH₂), 4.71 (2H, d J 5.89 Hz, NCH₂CH₂N), 5.30 (2H, t J 5.89 Hz, NCH₂CH₂N), 6.66 (1H, s, HC═CH), 6.93 (1H, s, HC═CH), 7.03(2H, d J 9.10 Hz, ArH), 7.31 (1H, s, N═CH), 7.43 (2H, m, ArH), 7.83 (1H, d J 8.03 Hz, ArH), 7.91 (2H, d, J 8.57 Hz, ArH), 8.06 (1H, d, J 8.57 Hz, ArH), 9.13 (1H, t J 5.89 Hz, NH); δ_{C} (75.5 MHz, DMSO-d₆) 168.51, 167.16, 166.62, 166.01, 147.34, 129.17, 129.17, 129.17, 128.30, 127.08, 125.87, 125.69, 122.25, 119.98, 119.34, 118.22, 113.57, 99.26, 55.31, 53.27, 45.60, 35.06; LC-MS-EI 444.5 (MH⁺, 100); Found (CI) 444.17997, C₂₃H₂₁N₇O₃ (M+H) requires 444.17841.

### EXAMPLE 44

### N-((5-(2-(2-(1H-imidazol-1-yl)ethyl)-2H-indazol-3-yl)-1,2,4-oxadiazol-3-yl)methyl)-4-(trifluoromethoxy)benzamide

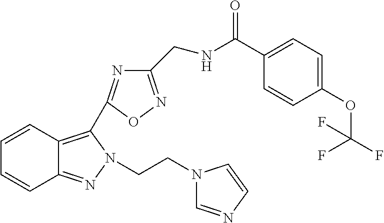

The above compound was prepared by analogous means to those used in the foregoing examples.

Orange powder (0.080 g, 20%); δ_{H} (500 MHz, DMSO-d₆) 4.60 (2H, t J 6.09 Hz, NCH₂), 4.75 (2H, d J 5.77 Hz, NCH₂CH₂N), 5.30 (2H, t J 6.06 Hz, NCH₂CH₂N), 6.66 (1H, s, HC═CH), 6.91 (1H, s, HC═CH), 7.31 (1H, s, N═CH), 7.46 (4H, m, ArH), 7.83 (1H, d J 8.57 Hz, ArH), 8.06 (3H, m, ArH), 9.40 (1H, t J 5.73 Hz, NH); δ_{C} (75.5 MHz, DMSO-d₆); LC-MS-EI 498.6 (MH⁺, 100); Found (CI) 498.15158 , C₂₃H₁₈F₃N₇O₃ (M+H) requires 498.15015.

### EXAMPLE 45

### (5-(2-(2-(1H-indazol-1-yl)ethyl)-5-bromo-2H-indazol-3-yl)-1,2,4-oxadiazol-3-yl)methanamine

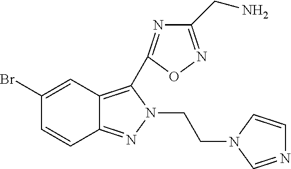

The above compound was prepared by analogous means to those used in the foregoing examples.

Beige solid (0.66 g, 50%); δ_{H} (300 MHz, DMSO-d₆) 4.50 (2H, bs, CH₂), 4.89 (2H, bs, NCH₂CH₂N), 5.43 (2H, bs, NCH₂CH₂N), 7.51 (1H, s, HC═CH), 7.59 (3H, m, ArH+HC═CH+N═CH), 7.80 (1H, d J 9.10 Hz, ArH), 8.34 (1H, m, ArH), 8.82 (2H, bs, NH₂); δ_{C} (75.5 MHz, DMSO-d₆); LC-MS-EI 390.2(MH⁺, 100); Found (FAB) 390.04863 C₁₅H₁₄BrN₇O (M+H) requires 390.04863.

### EXAMPLE 46

### N-((5-(2-(2-(1H-imidazol-1yl)ethyl)-5-bromo-2H-indazol-3-yl)-1,2,4-oxadiazol-3-yl)methyl)-2,3-dimethoxybenzamide

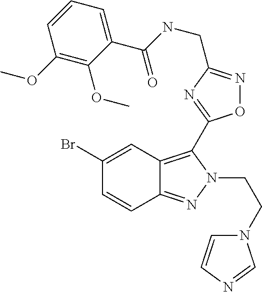

The above compound was prepared by analogous means to those used in the foregoing examples.

Orange solid (0.024 g, 50%); δ_{H} (300 MHz, MEOH-d₄) 3.86 (6H, m, OCH₃) 4.64 (2H, m, CH₂), 4.87 (2H, m, NCH₂CH₂N), 5.30 (2H, d J 5.54 Hz, NCH₂CH₂N), 6.71 (1H, s, HC═CH), 6.81 (1H, s, HC═CH), 7.15 (3H, m, ArH), 7.29 (1H, s, N═CH), 7.42 (1H, m, ArH), 7.61 (1H, m, ArH), 8.17 (1H, m, ArH); δ_{C} (75.5 MHz, MEOH-d₄); LC-MS-EI 554.5(MH⁺, 100); Found (FAB) 554.09809 C₂₄H₂₂BrN₇O₄ (M+H) requires 554.09744.

### EXAMPLE 47

### N-((5-(2-(2-(1H-imidazol-1-yl)ethyl)-5-bromo-2H-indazol-3-yl)-1,2,4-oxadiazol-3-yl)methyl)-3-(trifluoromethoxy)benzamide

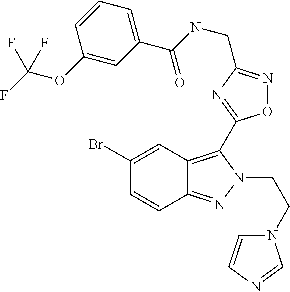

The above compound was prepared by analogous means to those used in the foregoing examples.

Orange solid (0.024 g, 50%); δ_{H} (300 MHz, MEOH-d₄) 4.65 (2H, t J 5.35 Hz, CH₂), 4.87 (2H, m, NCH₂CH₂N), 5.35 (2H, t J 5.35 Hz, NCH₂CH₂N), 6.70 (1H, t J 1.07 Hz, HC═CH), 6.79 (1H, t J 1.07 Hz, HC═CH), 7.27 (1H, m, N═CH), 7.50 (2H, m, ArH), 7.61 (1H, m, ArH), 7.69 (1H, d J 9.10 Hz, ArH), 7.83 (1H, d J 1.07 Hz, ArH), 7.93 (1H, m, ArH), 8.24 (1H, m, ArH); δ_{C} (75.5 MHz, MEOH-d₄); LC-MS-EI 578.4(MH⁺, 100); Found (EI) 577.05153 C₂₃H₁₇BrF₃N₇O₃ (M+H) requires 577.05025.

### EXAMPLE 48

### (5-(2-(2-(1H-imidazol-1-yl)ethyl)-4,6-dichloro-2H-indazol-3-yl)-1,2,4-oxadiazol-3-yl)methanamine

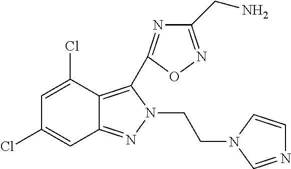

The above compound was prepared by analogous means to those used in the foregoing examples.

White solid (0.310 g, 19%); δ_{H} (300 MHz, MEOH-d₄) 4.48 (2H, m, CH₂), 5.00 (2H, m, NCH₂CH₂N), 5.27 (2H, m, NCH₂CH₂N), 7.47 (314, m, HC═CH+N═CH), 7.79 (1H, m, ArH), 8.81 (1H, m, ArH); δ_{C} (75.5 MHz, MEOH-d₄); LC-MS-EI 378.7(MH⁺, 100); Found (EI) 378.0637 C₁₅H₁₄Cl₂N₇O (M+H) requires 378.0635.

### EXAMPLE 49

### N-((5-(2-(2-(1H-imidazol-1-yl)ethyl)-4,6-dichloro-2H-indazol-3-yl)-1,2,4-oxadiazol-3-yl)methyl)-2,3-dimethoxybenzamide

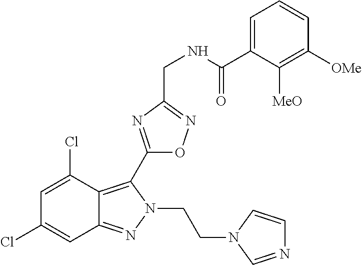

The above compound was prepared by analogous means to those used in the foregoing examples.

Yellow solid (0.290 g, 65%); δ_{H} (300 MHz, MEOH-d₄) 3.85 (3H, m, OCH₃), 3.91 (3H, s, OCH₃), 4.62 (2H, m, CH₂), 4.85 (2H, t J 5.54 Hz, NCH₂CH₂N), 5.11 (2H, t J 5.18 Hz, NCH₂CH₂N), 6.78 (1H, s, HC═CH), 6.83 (1H, s, HC═CH), 7.17 (3H, m, ArH), 7.37 (1H, s, N═CH), 7.42 (1H, m, ArH), 7.63 (1H, m, ArH), 8.17 (1H, m, ArH); δ_{C} (75.5 MHz, MEOH-d₄); LC-MS-EI 542.5(M⁺, 100); Found (EI) 542.1083 C₂₄H₂₂Cl₂N₇O₄ (M+H) requires 542.1110.

### EXAMPLE 50

### Biological Assays

Biological assays to determine sodium channel activity, neuroprotection and Naᵥ isoform selectivity were carried out according to the protocols set out in Clutterbuck et al (2009), J. Med. Chem. 52, 2694-2707.

The results of these assays are set out in the tables below.

Sodium channel activity

| Compound | Guanidine flux, IC₅₀ (μM) |
|---|---|
| Example 1 | 102.3 |
| Example 2 | 11 |
| Example 5 | 323.6 |
| Example 6 | 34 |
| Example 7 | 4 |
| Example 9 | 22 |
| Example 14 | 32 |

Neuroprotection

| Compound | Neuroprotection at 50 μM (%) |
|---|---|
| Example 2 | 35 |
| Example 5 | 60 |
| Example 6 | 89 |
| Example 7 | 62 |
| Example 9 | 41 |
| Example 12 | 43 |
| Example 14 | 80 |
| Example 15 | 46 |
| Example 16 | 37 |
| Example 17 | 52 |
| Example 18 | 37 |
| Example 24 | 41 |
| Example 25 | 57 |
| Example 24 | 57 |
| Example 27 | 38 |
| Example 29 | 40 |
| Example 30 | 71 |
| Example 31 | 39 |
| Example 41 | 54 |
| Example 42 | 63 |
| Example 43 | 55 |
| Example 44 | 31 |

Naᵥ isoform selectivity

| | Naᵥ isoform inhibition (%) | | | | | | | | |
|---|---|---|---|---|---|---|---|---|---|
| Compound | Naᵥ1.1 | Naᵥ1.2 EP | Naᵥ1.2 EP UD | Naᵥ1.3 | Naᵥ1.4 | Naᵥ1.5 | Naᵥ1.6 | Naᵥ1.7 | Naᵥ1.8 |
| Example 6 | 56 | 24.4 | 24.1 | 54 | 45 | 99 | 73 | 96 | 87 |
| Example 10 | 54 | | | 51 | 77 | 84 | 76 | 81 | 84 |
| Example 11 | 83 | | | 63 | 97 | 98 | 86 | 33 | 91 |
| Example 14 | 96 | 98.5 | not detected | 41 | 98 | 95 | 79 | 46 | 94 |
| Example 16 | 24 | 1.1 | 1.5 | 39 | 43 | 64 | 63 | 96 | 48 |
| Example 30 | 26 | 0.1 | 6.4 | 14 | 33 | 71 | 77 | 94 | 62 |

### EXAMPLE 51

### Neuroprotection of Compound of Example 15 in an Induced-Relapse During Experimental Autoimmune Encephalomyelitis (EAE)

Studies have indicated that sodium-channel blockers can be immunosuppressive in EAE (Lo et al. 2003, Bechtold et al. 2004, Black et al. 2006, Black et al. 2007). The immunosuppressive activity of Example 15 was investigated using a simple contact-hypersensitivity paradigm. Compared to vehicle-treatment, Example 15 failed to inhibit lymph node proliferative responses following daily i.p. administration of either 5 mg/kg, 25 mg/kg and 50 mg/kg i.p. from a day before sensitization to the time of collecting the lymph nodes. (Data not shown). Therefore, it was not necessary to dose-titrate Example 15 in order to avoid the immunosuppression-induced neuroprotection that would mask any real neuroprotective action of Example 15. In order to limit animal usage required for a full-dose-response, as proof-of-principle, 10 mg/kg of Example 15 i.p. was selected as an initial dose for study. This dose was within the range used in humans for sodium channel-blocking agents (1-50 mg/kg p.o.), particularly when scaled to mouse (USDHHS, 2005), and was well below the maximum tolerated dose (>50 mg/kg/daily). This was investigated for a neuroprotective potential in an induced-relapse during experimental autoimmune encephalomyelitis (EAE).

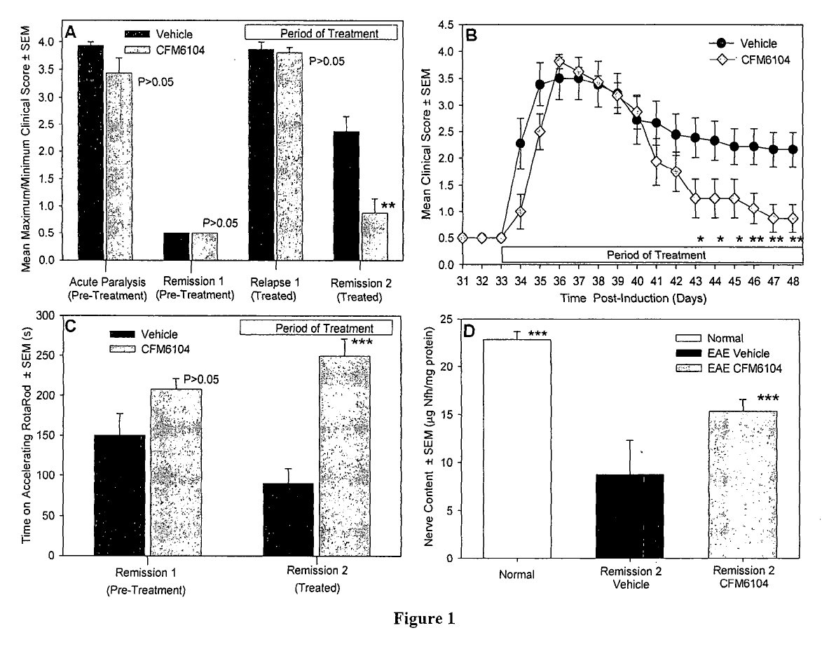

In many rodent models of MS, particularly during EAE the ABH mice, there is limited demyelination and nerve damage during the initial acute-phase of paralysis that occurs 2-3 weeks following sensitization (Baker et al. 1990, Jackson et al. 2005). Nerve damage and demyelination becomes more pronounced with disease duration and the development of relapsing disease (Baker et al. 1990). The neuroprotective potential of Example 15 was therefore examined in an induced-relapse EAE paradigm. This exhibits a highly synchronized and predictable relapse, about one week following induction in sensitized animals, and develops a more-consistent accumulation of neurological deficit/nerve loss compared to spontaneous, relapsing disease (Baker et al. 1990, O'Neill et al. 1991). The lack of immunosuppressive activity of Example 15 was indicated further by the observation that Example 15 did not significantly (P>0.05) inhibit the incidence, onset or maximum severity of relapsing EAE (FIGS. 1A, 1B). Although Example 15 did not inhibit the severity of paralysis during relapse (FIG. 1A), it did promote a significantly (P<0.01) greater degree of recovery of motor-function during remission (FIG. 1B). This could be seen not only using the subjective clinical scoring system (FIG. 1B) but importantly Example 15 significantly (P<0.001) reduced the loss of motor-function occurring as a consequence of relapse when assessed objectively using the rotarod (FIG. 1C). These improved motor outcomes were reflected by the capacity of Example 15 to reduce nerve loss, assessed by ELISA, occurring as a consequence of relapsing disease (FIG. 1D). Therefore Example 15 was exhibiting a neuroprotective effect and slows the loss of nerves that occurs as a consequence of inflammatory attack.

Results are shown in FIG. 1.

ABH mice were injected with 1 mg mouse spinal cord homogenate in Freunds adjuvant on days 0 and 7 p.i. to induce EAE and this was repeated on 27 p.i. to induce a relapse. Animals were injected daily i.p. with either vehicle or 10 mg/kg Example 15 from day 33 onwards. Animals were monitored for the development of clinical disease and the results represent: (A) The mean maximal clinical score, during the initial acute attack around day 15-20 and the clinical relapse around day 34-40, or the mean minimal clinical score during remission from paralytic attacks. (B) The mean daily clinical score following induction of relapse. (C) The time taken to fall from an accelerating rotorod was monitored pre-treatment on day 25 or on day 48 post-disease induction, when the experiment was terminated. (D) The spinal cords were removed and nerve content was measured using a neurofilament heavy chain (Nfh)-specific ELISA. The results represent the mean±SEM of 8 animals per group. *P<0.05, **P<0.01, ***P<0.001 compared to vehicle-treated animals.

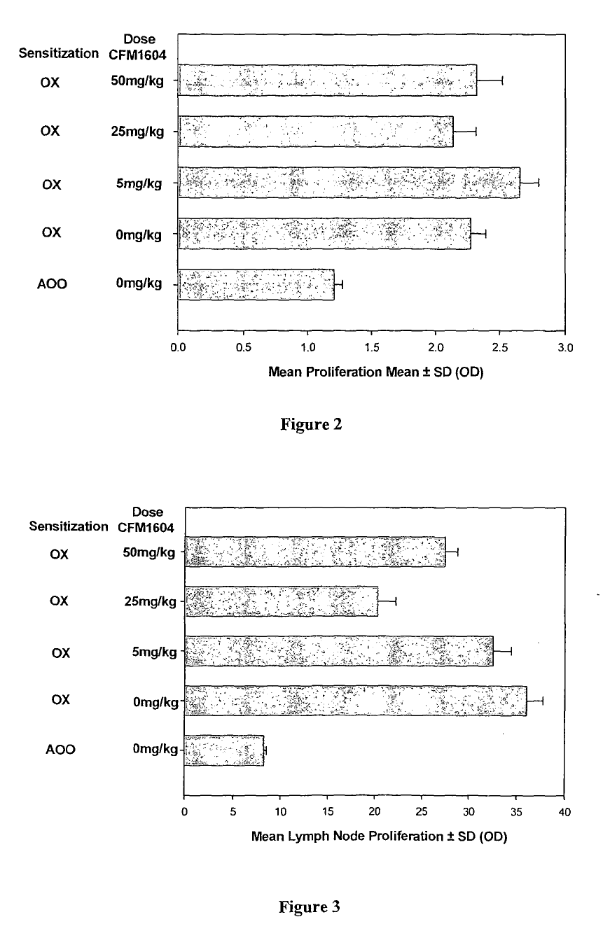

T cell proliferation per 5×10⁵ lymph node cells or proliferation adjusted for the number of cells in the lymph nodes is shown in FIGS. 2 and 3.

1. A method of blockading voltage-dependent sodium channels in a patient, which method comprises administering to the patient an indazole derivative of formula 1, or a pharmaceutically acceptable salt of N-oxide thereof, 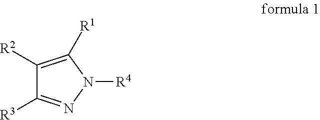
wherein:
either (a) R¹ and R² together, represent —(CR′)₄—, or an n-butylene or n-butenylene group, which group is unsubstituted or substituted by one or more R″ substituents, and R³ represents a moiety -A-L-Het-Y-R⁵ or -A-L-Het-H, or (b) R² and R³ together represent —(CR′)₄— or an n-butylene or n-butenylene group, which group is unsubstituted or substituted by one or more R″ substituents, and R¹ represents a moiety -A-L-Het-Y-R⁵ or -A-L-Het-H, wherein each R′ is the same, or different and represents hydrogen, halogen, C₁-C₆ alkyl, C₁-C₆ alkoxy, C₁-C₆ haloalkyl, C₁-C₆ haloalkoxy, nitro or cyano, and R″ is the same or different and represents halogen,
C₁-C₆ alkyl, C₁-C₆ alkoxy, C₁-C₆ haloalkyl, C₁-C₆ haloalkoxy, nitro or cyano; and
R⁴ represents -L′-A′,
wherein:
A and A′ are the same or different and each represent a 5- to 10-membered heteroaryl group or a 5- to 10-membered heterocyclyl group, which group is unsubstituted or substituted by one or more substituents selected from halogen, C₁-C₄ alkyl, C₁-C₄ alkoxy, C₁-C₄ haloalkyl, C₁-C₄ haloalkoxy, nitro and cyano substituents;
L and L′ are the same or different and each represent C₁-C₄ alkylene, C₂-C₄ alkenylene or C₂-C₄ alkynylene;
Het is —NR′″—, —O—, or —S—, wherein R′″ is hydrogen or C₁-C₄ alkyl;
Y is —CO—, —CO—O—, —CO—NR_{′}—, or —SO₂—, wherein R_{′} is hydrogen or C₁-C₄ alkyl;
R⁵ is C₁-C₆ alkyl, or, is a moiety which is a phenyl ring which is optionally fused to a further phenyl ring or to a 5- or 6-membered heteroaryl or heterocyclyl ring, which moiety is unsubstituted or substituted by one or more substituents selected from halogen, C₁-C₆ alkyl, C₁-C₆ alkoxy, C1-C6 haloalkyl, C₁-C₆ haloalkoxy, nitro, cyano, —NR_{′}—CO—R₄₁ , —CO—R_{″}, —O—CO—R_{″}, —CO—O—R_{′} and
—CO—N(R_{′})₂ substituents, wherein each R_{′} is the same or different and represents hydrogen or C₁-C₄ alkyl, and each R_{″} is the same or different and represents C₁-C₄ alkyl.

2. A The method according to claim 1 wherein the compound of formula 1 is a compound of formula (1a) or (1b), 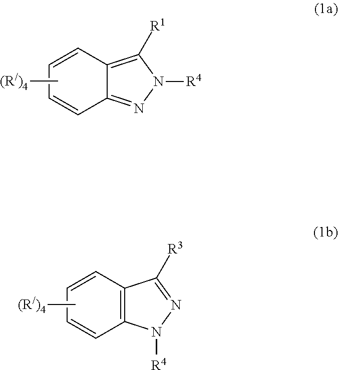
wherein R¹ and R³ represent a moiety -A-L-Het-Y-R⁵ or -A-L-Het-H, and R′, R⁴, A, L, Het, Y and R⁵ are as defined in claim 1.

3. The method according to claim 1 wherein each R′ is the same or different and represents hydrogen, halogen or C₁-C₄ alkoxy.

4. The method according to claim 1, wherein A is unsubstituted or substituted by 1 or 2 substituents selected from halogen, C₁-C₄ alkyl, C₁-C₄ alkoxy and C₁-C₄ haloalkyl substituents.

5. The method according to claim 1, wherein A is a 5 to 6 membered heteroaryl group.

6. The method according to claim 1, wherein L is a C₁-C₄ alkylene group.

7. The method according to claim 1, wherein Het is —NH—.

8. The method according to claim 1, wherein -Het-Y- is —NH—CO—, —NH—CO—O— or —NH—S(O)₂—.

9. The method according to claim 1, wherein when R₅ is a moiety which is a phenyl ring which is optionally fused to a further phenyl ring or to a 5- or 6-membered heteroaryl or heterocyclyl ring, said moiety is unsubstituted or substituted by 1, 2 or 3 substituents selected from halogen, C₁-C₄ alkyl, C₁-C₄ alkoxy, C₁-C₂ haloalkyl, C₁-C₂ haloalkoxy, nitro and —NR_{′}—CO—R_{″} substituents, wherein R_{′} is a hydrogen atom and R_{″} is C₁-C₄ alkyl.

10. The method according to claim 1, wherein when R⁵ is a C₁-C₆ alkyl group it is a t-butyl group, and when R⁵ is other than an alkyl group it is a phenyl, naphthyl, indolyl or benzodioxolyl group.

11. The method according to claim 1, wherein L′ is a C₁-C₄ alkylene group.

12. The method according to claim 1, wherein A′ is unsubstituted or substituted by 1, 2 or 3 C₁-C₄ alkyl groups

13. The method according to claim 1, wherein A′ is a 5- to 6-membered heteroaryl or 5- to 6-membered heterocyclyl group.

14. A method of blockading voltage-dependent sodium channels in a patient, which Method comprises administering to the patient an indazole derivative of formula (1a) or (1b), or a pharmaceutically acceptable salt or N-oxide thereof, 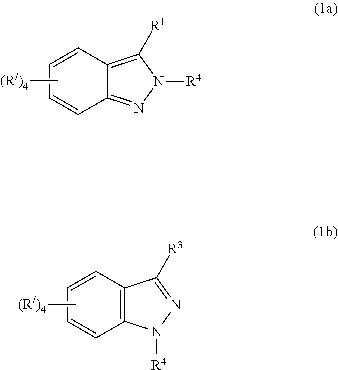
wherein:
R¹ and R³ represent a moiety -A-L-Het-Y-R⁵ or -A-L-Het-H;
each R′ is the same or different and represents hydrogen, fluorine, chlorine, bromine or methoxy, provided that 2, 3 or 4 of the R′ groups are hydrogen and, when two of the R′ groups are hydrogen atoms, the other two R_{′}R′ groups are chlorine atoms;
A is a 1,2,4-oxadiazolyl group;
L is methylene;
Het is —NH—;
Y is —CO—, —CO—O— or —S(O)₂—;
R⁵ is t-butyl or a phenyl, naphthyl, indolyl or benzodioxolyl group, which group is unsubstituted or substituted by 1, 2 or 3 substituents selected from methyl, methoxy, t-butyl, trifluoromethyl, trifluoromethoxy, fluoro, chloro, nitro and —NH—CO—CH₃ substituents; and
R⁴ represents -L′-A′, wherein L′ is methylene or ethlyene and A′ is a thiazolyl, pyridyl, imidazolyl or thienyl group, which group is unsubstituted or substituted by one methyl substituent.

15. The method according to claim 1, wherein the patient is suffering from or susceptible to normal tension glaucoma, multiple sclerosis; a motorneurone disease, stroke, spinal cord injury, Alzheimer's disease, Parkinson's disease or pain.

16. The method according to claim 15, wherein the patient is suffering from or susceptible to multiple sclerosis or pain.

17. An indazole derivative of formula 1, or a pharmaceutically acceptable salt or N-oxide thereof, 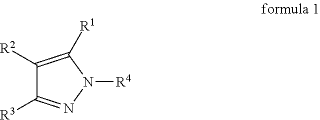
wherein:
either (a) R¹ and R² together represent —(CR′)₄—, or an n-butylene or n-butenylene group, which group is unsubstituted or substituted by one or more R″ substituents, and R³ represents a moiety -A-L-Het-Y-R⁵ or -A-L-Het-H, or (b) R² and R³ together represent —(CR′)₄— or an n-butylene or n-butenylene group, which group is unsubstituted or substituted by one or more R″ substituents, and R¹ represents a moiety -A-L-Het-Y-R⁵ or -A-L-Het-H, wherein each R′ is the same or different and represents hydrogen, halogen, C₁-C₆ alkyl, C₁-C₆ alkoxy, C₁-C₆ haloalkyl, C₁-C₆ haloalkoxy, nitro or cyano, and R″ is the same or different and represents halogen,
C₁-C₆ alkyl, C₁-C₆ alkoxy, C₁-C₆ haloalkyl, C₁-C₆ haloalkoxy, nitro or cyano; and
R⁴ represents -L′-A′,
wherein:
A and A′ are the same or different and each represent a 5- to 10-membered heteroaryl group or a 5- to 10-membered heterocyclyl group, which group is unsubstituted or substituted by one or more substituents selected from halogen, C₁-C₄ alkyl, C₁-C₄ alkoxy, C₁-C₄ haloalkyl, C₁-C₄ haloalkoxy, nitro and cyano substituents;
L and L′ are the same or different and each represent C₁-C₄ alkylene, C₂-C₄ alkenylene or C₂-C₄ alkynylene;
Het is —NR′″—, —O—, or —S—, wherein R′″ is hydrogen or C₁-C₄ alkyl;
Y is —CO—, —CO—O—, —CO—NR_{′}, or —SO₂—, wherein R_{′}, is hydrogen or C₁-C₄ alkyl;
R⁵ is C₁-C₆ alkyl, or is a moiety which is a phenyl ring which is optionally fused to a further phenyl ring or to a 5- or 6-membered heteroaryl or heterocyclyl ring, which moiety is unsubstituted or substituted by one or more substituents selected from halogen, C₁-C₆ alkyl, C₁-C₆ alkoxy, C₁-C₆ haloalkyl, C₁-C₆ haloalkoxy, nitro, cyano, —NR_{′}—CO—R_{″}, —O—CO—R_{″}, —CO—O—O—R_{′} and —CO—N(R_{′})₂ substituents, wherein each R_{′} is the same or different and represents hydrogen or C₁-C₄ alkyl, and each R_{″} is the same or different and represents C₁-C₄ alkyl.

18. An indazole derivative of formula (1a) or (1b), or a pharmaceutically acceptable salt of N-oxide thereof, 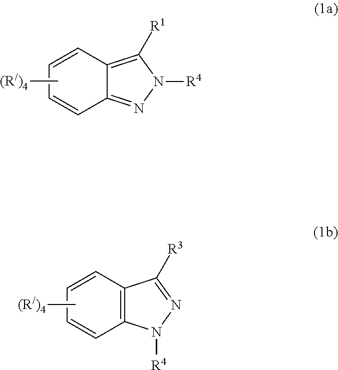
wherein R¹ and R³ represent a moiety -A-L-Het-Y-R⁵ or -A-L-Het-H, and R′, R⁴, A, L, Het, Y and R⁵ are as defined in claim 1.

19. A compound according to claim 18, wherein A is pyridyl, pyrazinyl, pyrimidinyl, pyridazinyl, thienyl, pyrazolidinyl, oxadiazolyl, oxazolyl, isoxazolyl, thiazolyl, thiadiazolyl, imidazolyl, pyridazolyl or pyrazolyl.

20. A compound according to claim 19 wherein A is oxadiazolyl.

21. A compound according to claim 20 wherein A is 1,2,4-oxadiazolyl.

22. (canceled)

23. (canceled)

24. A method according to claim 14, wherein the patient is suffering from or susceptible to normal tension glaucoma, multiple sclerosis, a motorneurone disease, stroke, spinal cord injury, Alzheimer's disease, Parkinson's disease or pain.

25. A method according to claim 14, wherein the patient is suffering from or susceptible to multiple sclerosis or pain.
